(19) United States
(12) Patent Application Publication
(10) Pub. No.: US 2025/0312309 A1
(43) Pub. Date: Oct. 9, 2025

(54) ALLYL-AND PROPARGYLAMINE-TYPE PHENETHYLAMINES AND TRYPTAMINES FOR TREATING MEDICAL DISORDERS

(71) Applicant: Mind Medicine, Inc., New York, NY (US)

(72) Inventors: Daniel TRACHSEL, Detligen (CH); Matthias Emanuel LIECHTI, Oberwil (CH); Felix LUSTENBERGER, Meggen (CH)

(73) Assignee: Mind Medicine, Inc., New York, NY (US)

(21) Appl. No.: 18/750,991

(22) Filed: Jun. 21, 2024

Related U.S. Application Data
(60) Provisional application No. 63/509,597, filed on Jun. 22, 2023.

(51) Int. Cl. A61K 31/4045; A61K 31/36; A61P 25/24

(52) U.S. Cl. CPC A61K 31/4045; A61K 31/36; A61P 25/24

(57) ABSTRACT

A composition of a compound represented by FIGS. 1A-1E for use in treating a medical disorder. A method of changing neurotransmission, by administering a pharmaceutically effective amount of composition to a mammal of a compound represented by FIGS. 1A-1E and inducing psychoactive effects in the mammal. A method of treating a patient having adverse reactions to psychedelics or entactogens, by administering a pharmaceutically effective amount of composition to the patient of a compound represented by FIGS. 1A-1E and avoiding adverse effects present with psychedelics or entactogens.

### BACKGROUND OF THE INVENTION

### 1. TECHNICAL FIELD

The present invention relates to both the substance definition and synthesis of phenethylamines, tryptamines and related compounds containing an allyl- and/or propargylamine moiety to be used in treating a medical disorder including as substance-assisted psychotherapy.

### 2. BACKGROUND ART

Psychedelics are substances inducing unique subjective effects including dream-like alterations of consciousness, affective changes, enhanced introspective abilities, visual imagery, pseudo-hallucinations, synesthesia, mystical-type experiences, ego-dissolution, and feelings of connectedness (M. E. Liechti, 2017; Passie, Halpern, Stichtenoth, Emrich, & Hintzen, 2008).

Psychedelics, mainly lysergic acid diethylamide (LSD) and psilocin, are currently investigated as potential medications. First clinical trials indicate potential efficacy of LSD and psilocybin in addiction (Bogenschutz, 2013; Bogenschutz et al., 2015; Bogenschutz et al., 2022; Johnson, Garcia-Romeu, Cosimano, & Griffiths, 2014; Johnson, Garcia-Romeu, & Griffiths, 2016; Krebs & Johansen, 2012), anxiety associated with life-threatening illness (Gasser et al., 2014; Gasser, Kirchner, & Passie, 2015; Holze, Gasser, Muller, Dolder, & Liechti, 2023), depression (R. Carhart-Harris et al., 2021; R. L. Carhart-Harris, Bolstridge, et al., 2016; Davis et al., 2021; Goodwin et al., 2022; R. R. Griffiths et al., 2016; Roseman, Nutt, & Carhart-Harris, 2017; Ross et al., 2016), and anxiety (R. R. Griffiths et al., 2016; Grob et al., 2011; Holze et al., 2023; Ross et al., 2016). Several trials investigating therapeutic effects of LSD, psilocybin, mescaline, and other psychedelics are also ongoing, as can be seen on www.clinicaltrials.gov. There is also evidence that the psychedelic brew Ayahuasca which contains the active psychedelic substance N,N-dimethyltryptamine (DMT) (Dominguez-Clave et al., 2016) can alleviate depression (Dos Santos et al., 2016; Palhano-Fontes et al., 2019; Sanches et al., 2016). DMT alone also showed antidepressant effects after a single administration in patients with depression (D′Souza et al., 2022).

Beside the psychedelics, a second class of compounds has been proven to be useful for substance-assisted psychotherapy, namely the entactogens. 3,4-Methylenedioxymethamphetamine (MDMA) is an example of this class of compounds and is a psychoactive drug that mainly alters mood inducing feelings of well-being, empathy, love, and trust. MDMA is investigated as an adjunct to psychotherapy for posttraumatic stress disorder (PTSD), social anxiety, autism (Danforth et al., 2018; Danforth, Struble, Yazar-Klosinski, & Grob, 2016; Mitchell et al., 2021; Mithoefer et al., 2019; Mithoefer, Wagner, Mithoefer, Jerome, & Doblin, 2010; Oehen, Traber, Widmer, & Schnyder, 2013), and is being studied and used for a range of other medical conditions. Such conditions where MDMA or related substances can be useful include, but are not limited to, substance-use disorder, depression, anxiety disorder, anxiety with life-threatening disease, personality disorder including narcistic and antisocial disorder, and obsessive-compulsive disorder. MDMA or related substances can also be used to enhance couple therapy. Substances related to MDMA can be chemically similar and/or pharmacologically similar and produce psychoactive effects that are like those of MDMA.

MDMA and pharmacologically related substances are thought to produce positive therapeutic long-term effects in the context of MDMA/substance-assisted psychotherapy by producing acute subjective positive mood effects that also enhance the effectiveness of psychotherapy and can be beneficial on their own. Such acute beneficial MDMA-effects include, but are not limited to, feelings of well-being, feelings of connectivity to others, feelings of increased trust, feelings of love, enhanced emotional empathy, and enhanced feelings of pro-sociality and prosocial behavior (Hysek et al., 2014).

Substances other than MDMA or other psychedelics than LSD or psilocybin can be more suitable with different therapeutic benefits/tolerability profiles. MDMA is the only entactogen currently investigated for substance-assisted psychotherapy. (Mitchell et al., 2021). Alternatives to MDMA have been suggested (Oeri, 2020). These alternative MDMA-like substances include many compounds that can share some similarity with MDMA based on their in vitro pharmacological profiles and based on reports of their subjective effects by recreational users (Oeri, 2020). Thus, “MDMA-like” refers to both a similar pharmacological profile to MDMA (Simmler et al., 2013) and/or a prodrug of MDMA or a related psychoactive substance and/or similar entactogenic or empathogenic subjective effects compared to MDMA (Dolder, Muller, Schmid, Borgwardt, & Liechti, 2018).

Although no psychedelic is currently licensed for medical use, psilocybin and LSD are already medically used in limited or compassionate use programs for example in Switzerland (Schmid, Gasser, Oehen, & Liechti, 2021). Existing psychedelics such as LSD, psilocybin, and DMT, or entactogens such as MDMA may not be suitable to be used in all patients considered for substance-assisted therapy. The availability of several substances with different properties is important and the present lack thereof is a therapeutic problem which will further increase with more patients needing psychedelic-assisted therapy and an increase in demand for such treatment once the efficacy of first treatments will be documented in large clinical studies. For example, some patients can react with strong adverse responses to existing therapies such as psilocybin presenting with untoward effects including headaches, nausea/vomiting, anxiety, cardiovascular stimulation, or marked dysphoria. Thus, novel compounds with psychedelic-like or entactogen-like or even mixed action are needed.

Psychedelic-like substances have also been developed that are assumed to be devoid of subjective psychedelic effects while they may still produce therapeutic effects in patients by enhancing neuroplasticity (Dong et al., 2021; Ly et al., 2018; Vargas et al., 2023)

For certain phenethylamines with an «-ethyl group, such as the compound Ariadne (2-amino-1-(2,5-dimethoxy-4-methylphenyl)-butane), a lack of psychedelic effects was documented, despite their high structural closeness to psychedelic phenethylamines (A. Shulgin & Shulgin, 1991), and possible mechanistic explanations have been given (Cunningham et al., 2023). The potential for therapeutical use of Ariadne has been investigated clinically, and treatment results were very promising and included rapid remission of psychotic symptoms in schizophrenics, relaxation in catatonics, complete remission of symptoms in Parkinson's disease (PD), and improved cognition in geriatric subjects (summarized in (Cunningham et al., 2023)). However, this compound has never entered the pharmaceutical market. Mechanistically, Ariadne and related compounds still behave as 5-HT2A receptor agonists, which is the primary target of psychedelic compounds, including many phenethylamines, tryptamines and ergolines. However, Ariadne and related compounds have shown to be substances with significantly lower signaling potency and efficacy in signaling pathways (Gq, G11, and β-arrestin2) coupled to 5-HT2A receptors in comparison to the prototypical psychedelic phenethylamine DOM (2,5-dimethoxy-4-methylamphetamine), which can explain the lack pf psychedelic effects (called as signaling efficacy hypothesis by the authors (Cunningham et al., 2023)). The non-psychedelic 5-HT2A receptor agonists thus represent another class of potential medications for use in neurological and psychiatric indications.

MDMA and related entactogens typically interact with monoamine transporters to inhibit monoamine uptake and to release serotonin, norepinephrine, and or dopamine (Simmler et al., 2013; Verrico, Miller, & Madras, 2007). Additionally, some entactogens interact with mono amine oxidase (MAO) and act as MAO inhibitors. Furthermore, some entactogens like MDMA interact with the vesicular monoamine transporter (VMAT). MAO inhibitors are used as effective antidepressants and novel MDMA-like substances that inhibit MAO may also have additional antidepressant properties. Thus, compounds that are like MDMA and inhibit MAO in addition to other pharmacological effects are promising medications to treat depression and other disorders where MAO inhibitors are effective.

LSD, psilocybin, mescaline, and DOM are all thought to induce their acute psychedelic effects primarily via their common stimulation of the 5-HT2A receptor. All serotonergic psychedelics including LSD, psilocybin, DMT, mescaline, and DOM are agonists at the 5-HT2A receptor (Rickli, Moning, Hoener, & Liechti, 2016) and produce overall largely similar effects (Holze et al., 2022; Snyder, Faillace, & Hollister, 1967). However, there are differences in the receptor activation, receptor subtype selectivity profiles and in the subsequent signal transduction pathway activation patterns between the substances that can induce different subjective effects. LSD potently stimulates the 5-HT2A receptor but also 5-HT2B/C, 5-HT1 and D1-3 receptors (Rickli et al., 2016). Psilocin, i.e., the active metabolite derived from the prodrug psilocybin in the human body, also stimulates the 5-HT2A receptor but additionally inhibits the 5-HT transporter (SERT) (Rickli et al., 2016). Mescaline binds in a similar, rather low concentration range to 5-HT2A, 5-HT2B, 5-HT2C, 5-HT1A and a2A receptors (Rickli et al., 2016). DOM shows high affinities at the 5-HT2A and 5-HT2C receptors (Braden & Nichols, 2007; Glennon, Raghupathi, Bartyzel, Teitler, & Leonhardt, 1992) but very low affinity at the 5-HT1A receptor (Janowsky et al., 2014). In contrast to LSD, psilocybin and mescaline and DOM show no affinity for D2 receptors (Rickli et al., 2016). Thus, LSD exhibits greater dopaminergic activity than psilocybin, mescaline, and DOM, and psilocin (psilocybin) also exhibits an action at the SERT similar, although to a lesser extent, to MDMA. DOM and its related psychedelic phenethylamines do not interact with the SERT in contrast to psilocin (data on file). Taken together, the pharmacological profiles of LSD, psilocin, and mescaline (as well as DOM) show some differences, and these differences may translate into slightly different psychoactive profiles and differences in therapeutic effects and tolerability in humans.

The acute subjective effects of psychedelics are mostly positive in most humans (R. L. Carhart-Harris, Kaelen, et al., 2016; Dolder, Schmid, Mueller, Borgwardt, & Liechti, 2016; Dolder et al., 2017; Holze et al., 2019; Holze et al., 2022; Holze, Vizeli, et al., 2021; Schmid et al., 2015). However, there are also negative subjective effects such as anxiety in many humans likely depending on the dose used, personality traits (set), the setting (physical and social environment) and other factors. The induction of an overall positive acute response to the psychedelic is critical because several studies showed that a more positive experience is predictive of a greater therapeutic long-term effect of the psychedelic (R. R. Griffiths et al., 2016; Holze et al., 2023; Roseman et al., 2017; Ross et al., 2016). Even in healthy subjects, a more positive acute response to a psychedelic including LSD has been shown to be linked to more positive long-term effects on well-being (R. Griffiths, Richards, Johnson, McCann, & Jesse, 2008; Schmid & Liechti, 2018).

Entactogens exert their pharmacological action primarily by an acute release and reuptake inhibition of monoamines and by additional target interaction in a “golden” ratio. The relative dopamine over serotonin transporter interaction (DAT/SERT) potency ratio is a key determinant of the type of psycho-activity produced by compound: substances with a low DAT/SERT-ratio (<1) are MDMA-like entactogenic compounds while substances with a high DAT/SERT-ratio (>10) and therefore a predominant dopaminergic action are amphetamine and methamphetamine-like stimulants (M. Liechti, 2015; Simmler et al., 2013). Additionally, MDMA also releases oxytocin and oxytocin partly contributes to the subjective and possibly also therapeutic effects of MDMA (Dumont et al., 2009; Francis, Kirkpatrick, de Wit, & Jacob, 2016; Holze, Avedisian, Varghese, Eckert, & Liechti, 2021; Hysek et al., 2014). MDMA and related substances are thought to produce positive therapeutic long-term effects in the context of MDMA/substance-assisted psychotherapy by producing acute subjective positive mood effects that also enhance the effectiveness of psychotherapy and can be beneficial on their own. Such acute beneficial MDMA-effects include, but are not limited to, feelings of well-being, feelings of connectedness to others, feelings of increased trust, feelings of love, enhanced emotional empathy, and enhanced feelings of pro-sociality and prosocial behavior (Holze et al., 2020; Hysek et al., 2014; Kirkpatrick, Lee, Wardle, Jacob, & de Wit, 2014; Schmid et al., 2014).

Known psychedelics and entactogens have relevant acute side effects to different degrees depending on the subject treated including increased blood pressure, increased heart rate, nausea and vomiting, negative body sensations, anxiety, emotional distress, and dysphoria as well as headaches. Such side effects of a substance are often linked to its interactions with pharmacological targets. For example, interactions with adrenergic receptors can result untoward clinical cardio-stimulant properties. Additionally, changes in the relative activation profile of serotonin 5-HT receptors, monoamine transporters and other targets change the quality of the psychoactive effects. Alterations in the binding potency, the binding mode, and the potency in activating the subsequent signaling pathways at 5-HT2A receptors, monoamine transporter interaction profile as well as the molecule's lipophilicity can mostly determine the clinical dose to induce psychoactive effects. Alterations changing the metabolic stability of the compounds can also change the duration of action of the substance significantly. Some of the known compounds, such as LSD, for substance-assisted psychotherapy can be unfavorable in some patients with its long duration of action of 8-14 hours. Additionally, such long therapeutic sessions can significantly contribute to the overall therapeutic costs.

New compounds are needed to provide substances with an improved effect profile such as, but not limited to, more positive effects, less adverse effects, different qualitative effects, and shorter duration of acute effect, or with less acute effects but with long-term benefits.

### SUMMARY OF THE INVENTION

The present invention provides for a composition of a compound represented by FIGS. 1A-1E for use in treating medical disorders, wherein R_{α1} and R_{α2} is, independently and in any combination, hydrogen, deuteron, C₁-C₆ saturated and unsaturated alkyl optionally deuterated or fluorinated, C₃-C₆ saturated and unsaturated cycloalkyl-(C₁-C₆)alkyl optionally deuterated or fluorinated, and R_{α1} and R_{α2} can be combined to form a cyclic moiety such as cycloalkyl, oxacycloalkyl, thiacycloalkyl or azacycloalkyl, which can be further substituted in any combination with deuteron, fluorine, alkyl, alkenyl or alkynyl substituents, and
R1, R2, R5 and R6 are, independently and in any combination hydrogen, deuteron, or fluorine; or
C₁-C₆ branched or unbranched alkyl with the alkyl optionally and independently substituted with F₁-F₁₃ fluorine and/or D₁-D₁₃ deuteron substituents; or
C₃-C₆ cycloalkyl optionally and independently substituted with one or more substituents such as F₁-F₁₁ fluorine and/or D₁-D₁₁ deuteron and/or C₁-C₂ alkyl; or
(C₃-C₆ cycloalkyl)-C₁-C₂ branched or unbranched alkyl optionally and independently substituted with one or more substituents such as F₁-F₁₇ fluorine and/or D₁-D₁₇ deuteron and/or C₁-C₂ alkyl; or
C₂-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₆ branched or unbranched alkynyl where any of the carbons of the branched or unbranched alkynyl substituent is optionally substituted independently and in any combination with one or more C₁-C₂ alkyl, with F₁-F₉ fluorine, with D₁-D₉ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₃-C₆ branched or unbranched alkoxyalkyl, alkoxyalkenyl or alkoxyalkynyl optionally and independently substituted with F₁-F₁₃ fluorine and/or D₁-D₁₃ deuteron substituents; or
any halogen; or
a nitrogen-containing substituent such as CN or NO₂; and furthermore
R3 and R4 are, independently and in any combination, hydrogen; or
C₁-C₃ branched or unbranched alkyl with the alkyl optionally and independently substituted with F₁-F₇ fluorine and/or D₁-D₇ deuteron substituents or with aryl or heteroaryl bearing no or up to any number of ether, thioether, halogen, alkyl, fluorinated alkyl, alkenyl, alkynyl or nitrogen-containing substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₆ branched or unbranched alkyl optionally and independently substituted with one or more substituents such as F₁-F₁₅ fluorine and/or D₁-D₁₅ deuteron and/or C₁-C₂ alkyl; or
C₃-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₅ fluorine, with D₁-D₁₅ deuteron, with C₂ alkenyl; or
C₃-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl; or
are combined to form a cyclic moiety such as C₃-C₆ cycloalkyl, oxacycloalkyl, thiacycloalkyIS or azacycloalkyl, which can be further substituted in any combination with deuteron, fluorine, alkyl, alkenyl or alkynyl substituents; and furthermore

A represents aryl, wherein the aryl is independently di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro, iodo;
or A represents an indole-, benzo [1,3] dioxolyl-, 1,3-benzoxathiolyl-, a 1,3-benzodithiolyl-, a 2,3-dihydrobenzofuranyl, a 2,3-dihydrobenzo [b] thienyl- or a benzothioenyl group wherein said groups are independently attached and are independently unsubstituted, mono-, di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro or iodo.

The present invention provides for a composition of a compound particularly represented by FIGS. 2A-2H for use in treating medical disorders.

The present invention provides a method of changing neurotransmission, by administering a pharmaceutically effective amount of a compound of FIGS. 1A-1E and particularly of a compound represented by FIGS. 2A-2H to a mammal, interacting with serotonin 5-HT2A receptors and/or with monoamine transporters and/or with amine oxidases in the mammal, and inducing psychoactive effects.

The present invention also provides for a method of treating a patient having adverse reactions to psychedelics or entactogens by administering phenethylamines, tryptamines or related derivatives containing an allyl- and/or propargylamine moiety as represented in FIGS. 1A-1E and particularly in FIGS. 2A-2H to the patient and avoiding adverse effects present with other psychedelics or entactogens.

The present invention also provides for a method of treating a patient by administering phenethylamines, tryptamines or related derivatives containing an allyl- and/or propargylamine moiety as represented in FIGS. 1A-1E and particularly in FIGS. 2A-2H to the patient for cognitive enhancing effects or mood enhancing effects.

The present invention also provides for a method of changing neurotransmission of an individual, by administering phenethylamines, tryptamines or related derivatives containing an allyl- and/or propargylamine moiety as represented in FIGS. 1A-1E and particularly in FIGS. 2A-2H and changing neurotransmission in the individual.

### DESCRIPTION OF THE DRAWINGS

Other advantages of the present invention are readily appreciated as the same becomes better understood by reference to the following detailed description when considered in connection with the accompanying drawings wherein:

FIG. 1A shows the chemical structure of allylamine-type compounds of invention, FIG. 1B shows the chemical structure of allylamine-type compounds of invention, FIG. 1C shows the chemical structure of propargylamine-type compounds of invention, FIG. 1D show the chemical structure of allylamine-type compounds of invention, and FIG. 1E shows the chemical structure of allyl-propargylamine-type compounds of invention;

FIG. 2A show compound 8 as an example of allylamine-type derivatives represented by FIG. 1A; FIG. 2B shows compound 24 as an example of allylamine-type derivatives represented by FIG. 1B, FIG. 2C shows compound 25 as an example of allylamine-type derivatives represented by FIG. 1B, FIG. 2D shows compound 27 as an example of propargylamine-type derivatives represented by FIG. 1C, FIG. 2E shows compound 28 as an example of propargylamine-type derivatives represented by FIG. 1C, FIG. 2F shows compound 35 as an example of allylamine-type derivatives represented by FIG. 1B, FIG. 2G shows compound 37 as an example of propargylamine-type derivatives represented by FIG. 1C, FIG. 2H shows compound 43 as an example of allylamine-type derivatives represented by FIG. 1B;

FIG. 3 summarily describes the generic synthetic route to allylamine-type derivatives represented by FIG. 1A;

FIG. 4 summarily describes the synthetic route to the representative compound 8;

FIG. 5 summarily describes the generic synthetic route to allylamine- and propargylamine-type derivatives represented by FIG. 1B;

FIG. 6 summarily describes the synthetic route to the representative compounds 24, 25, 27 and 28;

FIG. 7 summarily describes the synthetic route to the representative compounds 35 and 37;

FIG. 8 summarily describes the synthetic route to the representative compound 43;

FIG. 9 shows screening results of compounds of FIGS. 2A-2F and 2H in their abilities to inhibit serotonin (5-HT) uptake at 1 μM and 10 μM concentrations;

FIG. 10 shows screening results of compounds of FIGS. 2A-2F and 2H in their abilities to form inositol monophosphate (IP1) at the 5-HT2A receptor, at 1μM and 10μM concentrations, together with the comparators serotonin (5-HT), MDA and mescaline; and

FIG. 11 shows a comparison of screening results of compounds of FIGS. 2F and 2G in their abilities to form inositol monophosphate (IP1) at the 5-HT2A receptor in comparison to serotonin (5-HT).

### DETAILED DESCRIPTION OF THE INVENTION

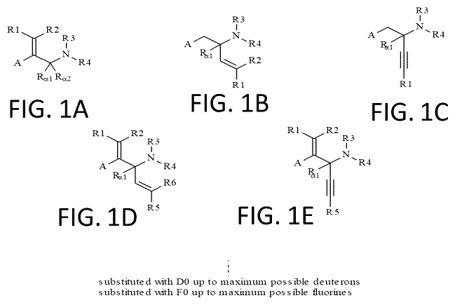

The present invention provides for a composition of a compound represented by FIGS. 1A-1E for use in treating medical disorders, and especially in substance-assisted therapy, wherein:
R_{α1} and R_{α2} is, independently and in any combination, hydrogen, deuteron, C₁-C₆ saturated and unsaturated alkyl optionally deuterated or fluorinated, C₃-C₆ saturated and unsaturated cycloalkyl-(C₁-C₆)alkyl optionally deuterated or fluorinated, and R_{α1} and R_{α2} can be combined to form a cyclic moiety such as cycloalkyl, oxacycloalkyl, thiacycloalkyl or azacycloalkyl, which can be further substituted in any combination with deuteron, fluorine, alkyl, alkenyl or alkynyl substituents, and
R1, R2, R5 and R6 are, independently and in any combination hydrogen, deuteron, or fluorine; or
C₁-C₆ branched or unbranched alkyl with the alkyl optionally and independently substituted with F₁-F₁₃ fluorine and/or D₁-D₁₃ deuteron substituents; or
C₃-C₆ cycloalkyl optionally and independently substituted with one or more substituents such as F₁-F₁₁ fluorine and/or D₁-D₁₁ deuteron and/or C₁-C₂ alkyl; or
(C₃-C₆ cycloalkyl)-C₁-C₂ branched or unbranched alkyl optionally and independently substituted with one or more substituents such as F₁-F₁₇ fluorine and/or D₁-D₁₇ deuteron and/or C₁-C₂ alkyl; or
C₂-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₆ branched or unbranched alkynyl where any of the carbons of the branched or unbranched alkynyl substituent is optionally substituted independently and in any combination with one or more C₁-C₂ alkyl, with F₁-F₉ fluorine, with D₁-D₉ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₃-C₆ branched or unbranched alkoxyalkyl, alkoxyalkenyl or alkoxyalkynyl optionally and independently substituted with F₁-F₁₃ fluorine and/or D₁-D₁₃ deuteron substituents; or
any halogen; or
a nitrogen-containing substituent such as CN or NO₂; and furthermore
R3 and R4 are, independently and in any combination, hydrogen; or
C₁-C₃ branched or unbranched alkyl with the alkyl optionally and independently substituted with F₁-F₇ fluorine and/or D₁-D₇ deuteron substituents or with aryl or heteroaryl bearing no up to any number of ether, thioether, halogen, alkyl, fluorinated alkyl, alkenyl, alkynyl or nitrogen-containing substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₆ branched or unbranched alkyl optionally and independently substituted with one or more substituents such as F₁-F₁₅ fluorine and/or D₁-D₁₅ deuteron and/or C₁-C₂ alkyl; or
C₃-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₅ fluorine, with D₁-D₁₅ deuteron, with C₂ alkenyl; or
C₃-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl; or
are combined to form a cyclic moiety such as C₃-C₆ cycloalkyl, oxacycloalkyl, thiacycloalkyl or azacycloalkyl, which can be further substituted in any combination with deuteron, fluorine, alkyl, alkenyl or alkynyl substituents; and furthermore

A represents aryl, wherein the aryl is independently di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro, iodo;
or A represents an indole-, benzo[1,3]dioxolyl-, 1,3-benzoxathiolyl-, a 1,3-benzodithiolyl-, a 2,3-dihydrobenzofuranyl, a 2,3-dihydrobenzo [b]thienyl- or a benzothioenyl group wherein said groups are independently attached and are independently unsubstituted, mono-, di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro or iodo.

In addition to the aforementioned description of compounds represented by FIGS. 1A-1E, any non-protic hydrogen can be replaced by a deuteron or a fluorine in any combination. Specifically, this complements to compounds represented by FIGS. 1A-1E by bearing one deuteron up to a completely deuterated compound or bearing one fluorine up to a completely fluorinated compound, with any combination of deuteron and fluorine, and any stereoisomers thereof. More specifically, a deuteration and/or fluorination can be conducted, independently and in any combination, on any of the substituent groups attached, on the aromatic nucleus, or on the alkyl-, alkenyl- or alkynylamine sidechain.

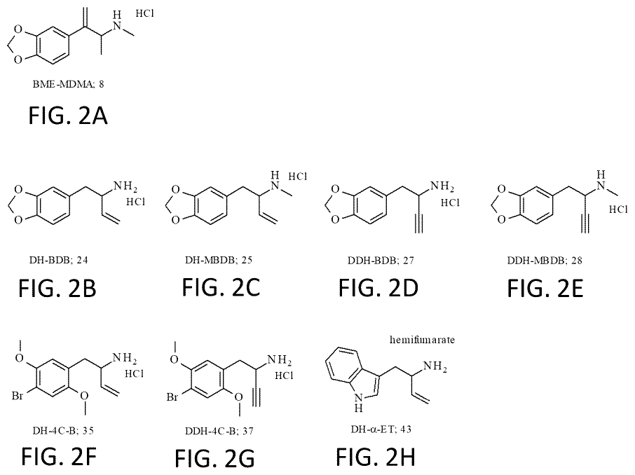

The present invention provides for a composition of a compound particularly represented by FIGS. 2A-2H for use in treating medical conditions and especially substance-assisted therapy.

The compounds represented by FIGS. 1A-1E and 2A-2H are basic compounds which form acid addition salts with inorganic or organic acids. Therefore, they form pharmaceutically acceptable inorganic and organic salts with pharmacologically acceptable inorganic or organic acids. Acids to form such salts can be selected from inorganic acids such as hydrochloric acid, hydrobromic acid, hydroiodic acid, sulfuric acid, nitric acid, phosphoric acid, and the like, and organic acids, such as carbonic acid, p-toluenesulfonic acid, methanesulfonic acid, oxalic acid, succinic acid, citric acid, benzoic acid, and the like. Thus, examples of such pharmaceutically acceptable salts are sulfate, pyrosulfate, bisulfate, sulfite, bisulfite, phosphate, monohydrogen-phosphate, dihydrogenphosphate, metaphosphate, pyro-phosphate, chloride, bromide, iodide, formate, acetate, propionate, decanoate, caprylate, acrylate, isobutyrate, caproate, heptanoate, oxalate, malonate, succinate, suberate, sebacate, fumarate, maleate, benzoate, phthalate, sulfonate, phenylacetate, citrate, lactate, glycollate, tartrate, methanesulfonate, propanesulfonate, mandelate and the like. Preferred pharmaceutically acceptable salts are those formed with hydrochloric acid and fumaric acid.

Furthermore, and without loss of generality and elaborating on details, the invention includes any prodrugs, i.e., any chemical modification of the described compounds that is (metabolically) converted to the described compound in the human body. It can be any prodrug that relies on enzymatic activation and/or that takes advantage of physiological chemical conditions for release of the drug. The basic amino function of the phenethylamine can chemically be transformed to any suitable prodrug that liberates the parent drug. Examples, but not limited to those, are amide, carbamate, ureate, N-Mannich base, amino sugars, imines (Schiff bases), enamines, enaminones and THTT. Using a prodrug allows for improving how an active drug is absorbed, distributed, metabolized, and excreted. Prodrugs can be used to prevent release of the active drug in the gastrointestinal tract upon administration so that the drug can be released more favorably elsewhere in the body or to cause an extended, prolonged release.

The general chemical terms used for FIGS. 1A-1E have their usual meanings. For example, the term “alkyl” includes unbranched as well as branched alkyl groups, such as methyl, ethyl, n-propyl, isopropyl, n-butyl, isobutyl, sec-butyl, tert-butyl, and the like. For another example, the term “cycloalkyl” includes such groups as cyclopropyl, cyclobutyl, cyclopentyl, and the like. For yet another example, the term “alkylcycloalkyl” is used as “cycloalkylalkyl” and includes such groups as consisting of an alkyl as outlined before, coupled with a cycloalkyl as outlined before. Some examples include cyclopropylmethyl, cyclopropylethyl, cyclobutylmethyl, cyclopentylmethyl, (2-methylcyclopropyl) methyl, and the like. The term “oxacycloalkyl” includes such groups as oxetanes, tetrahydrofuranes, and the like. The term “thiacycloalkyl” includes groups such as thietanes, and the like. The term “azacycloalkyl” includes azetidines, pyrrolidines, and the like. Further on, the term “alkenyl” includes unbranched as well as branched alkenyl groups, and includes such groups as vinyl (ethenyl), 1-propenyl, 2-propenyl, isopropenyl, 1-butenyl, 2-butenyl, 3-butenyl and the like, with a configuration of cis, trans, E, or Z, in any combination or purity. Further on, the term “alkenyl” also includes alkylidenes such as methylidene, ethylidene and alike. Thus, the number one in “C₁-C₃ alkenyl” or in “C₁-C₃ alkenyl” can be used for methylidene, e.g., when a H₂C=group is attached to a cyclus or to a chain. The term “alkylalkenyl” consists of any combination and branching of an alkenyl group with an alkyl group, with a configuration of cis, trans, E, or Z, in any combination or purity. Examples for such terms are 1-prop-2-enyl, 2-prop-1-enyl, 1-but-2-enyl, 1-but-3-enyl, 1-methyl-1-prop-2-enyl and the like. The term “alkynyl” includes unbranched as well as branched alkynyl groups, and includes groups such as ethynyl, 1-propyn-1-yl, 1-propyn-3-yl, 3-propynyl, 1-butynyl, 2-butynyl, 3-butynyl, phenylethynyl, and the like. The term “alkylalkynyl” consists of any combination and branching of an alkynyl group with an alkyl group. The term “alkylene” defines any unbranched or branched alkyl group serving as a connection between two molecular entities, substituents, or groups, or as an entity allowing to build a cyclic setup together with the molecular entity, substituent, or group. For some examples, methylene, ethylene, propylene or methylpropylene are included, and as cycles, e.g., aziridines, azetidines, oxetanes are some examples. An “aryl” group, alone or in combination, is defined as a substituent that contains one or more aromatic (annulated) homocycles, such as phenyl or naphthyl and can be unsubstituted or substituted. A “heteroaryl” group is defined as any aromatic ring system containing a conjugate pi electron system causing aromaticity, such as thiophene, furane, pyrrole, selenophene, pyrazole, oxazole, thiazole, isoxazole, isothiazole, benzothiophene, benzofurane, pyridine, pyrimidine, pyrazine, and the like. Such heteroaryl groups can further be annulated. A benzyl substituent defines a phenylmethyl group that can bear none or any numbers of substituents on the methylene or phenyl unit such as deuteron, fluorine, chlorine, bromine, iodine, methyl, ethyl, methoxy, methylthio, hydroxy, nitrile, methylenedioxy and the like. Such benzyl groups can further be annulated. A Heteroarylmethyl consists of a heteroaryl group as defined before attached to a methylene unit and can bear none or any numbers of substituents on the methylene or phenyl unit such as deuteron, fluorine, chlorine, bromine, iodine, methyl, ethyl, methoxy, methylthio, hydroxy, nitrile, methylenedioxy and the like. Such heteroarylmethyl groups can further be annulated. The term “unsaturated” used for alkanes, alkyl, cycloalkanes and cycloalkyl includes alkenes, alkynes, cycloalkenes and cycloalkynes. The term “halogen” includes a fluorine, chlorine, bromine, and iodine substituent, and the number of halogens can be one to as much as chemically possible which corresponds to a completely halogenated substituent, also known under the term “polyhalogenated.” The term “deuterated” includes numbers of deuteron atoms that can be one to as much as chemically possible which corresponds to a completely deuterated substituent, also known under the term “polydeuterated”. Any ratios and additional stereoisomers caused by introduction of fluorine and/or deuteron atoms are included. Terms such as “F0 to F11 fluorinated” or “D0 to D5 deuterated” correspond to non-fluorinated up to undeca-fluorinated (eleven fluorine atoms), and non-deuterated to penta-deuterated (five deuterons), respectively. Similarly, this is also given with a term, as an example, “F₀-F₁₁ fluorine”, which means that the substituent can also contain zero fluorine and thus be non-fluorinated. The term “Aₘ-Aₙ”—m and n being a number from zero to 99 and indicating the amount of atoms A—is descriptive for the number of atoms A of a given group or substituent as a sum. An example for such terms is C₃-C₆ cycloalkylalkyl and means that it can include a cyclopropyl, a cyclobutyl, a cyclopropylmethyl or a cyclobutylethyl or any other cycloalkylalkyl group consisting of three to six carbons. In a similar way, as an example, “C₁₋₃—O—C₁₋₃” is descriptive for an alkoxyalkyl group consisting of an alkyl group with one to three carbons attached to an Oxygen attached itself to an alkyl group with one to three carbons. Such a representative alkoxyalkyl group can be, as examples, methoxymethyl, methoxyethyl, ethoxymethyl and alike. The counting number of atoms or substituents can either be shown with normal characters or with subscripted characters. Thus, as an example, “C₁₋₃—O—C₁₋₃” is being used equally to “C1-3—O—C1-3”.

Those skilled in the art will appreciate that certain of the compounds of the present invention have at least one chiral carbon, and can therefore exist as a racemate, as individual enantiomers or diastereomers, and as mixtures of individual enantiomers or diastereomers in any ratio. For example, individual enantiomers of compounds of the invention are illustrated in FIG. 1A where R_{α1} is different from R_{α2} or in FIGS. 1B-1E where R_{α1} is different from any of the three remaining substituents attached to the carbon bearing R_{α1}. Those skilled in the art will also appreciate that those compounds of the invention where a substituent of a compound in FIGS. 1A-1E includes a substituent allowing configurational isomerism, can bear additional isomers. While it is a preferred embodiment of the invention that the compounds of the invention exist are used as racemates or mixtures of diastereomers, the present invention also contemplates the compounds of the invention existing in individual enantiomeric or diastereomeric pure form.

Those skilled in the art will also appreciate that certain of the compounds of the present invention have at least one double bond leading, depending on the double bond's substituents, to cis/trans or EIZ configurational isomerism. While it is a preferred embodiment of the invention that the compounds of the invention exist are used as pure configurational isomers, the present invention also contemplates the compounds of the invention existing in individual cis/trans or EIZ mixtures, respectively.

The individual enantiomers and diastereomers can be prepared by chiral chromatography of the racemic or enantiomerically or diastereomerically enriched amine, or fractional crystallization of salts prepared from racemic- or enantiomerically- or diastereomerically-enriched amine and a chiral acid. Alternatively, the free amine can be reacted with a chiral auxiliary and the enantiomers or diastereomers separated by chromatography or crystallization followed by removal of the chiral auxiliary to regenerate the free amine. Furthermore, separation of enantiomers or diastereomers can be performed at any convenient point in the synthesis of the compounds of the invention. The compounds of the invention can also be prepared by application of chiral syntheses. The compound itself is a pharmacologically acceptable acid addition salt thereof.

The individual cis/trans or EIZ configurational isomers can be accessed by either selective synthesis or by separation techniques addressing the different physicochemical properties of the configurational isomers by applying techniques such as chromatography, crystallization, distillation, or extraction.

In patients that have adverse reactions to other psychedelics or entactogens, allyl- or propargylamine-type phenethylamines and tryptamines and related compounds can be useful as alternative treatments. In some patients, allyl- or propargylamine-type phenethylamines and tryptamines and related compounds can also be useful because another experience than made with known compounds such as DOM, mescaline, psilocybin, LSD, DMT or MDMA is necessary or because a patient is not suited for therapy with these existing approaches a priori. Thus, allyl- or propargylamine-type phenethylamines and tryptamines and related compounds of subfigures FIGS. 1A-E can serve as alternative treatment options with characteristics sufficiently similar to other psychedelics and entactogens to be therapeutic but also sufficiently different to provide added benefits or avoid negative effects of other psychedelics or entactogens.

Based on structural relations, the compounds of FIGS. 1A-1E described in the present invention are expected to have overall comparable pharmacological properties to phenethylamine- or tryptamine-based psychedelics or entactogens as described above. However, some of the compounds can also act as more discrete compounds to be used in therapy and substance-assisted psychotherapy and to enhance cognition and/or mood when used at lower doses and without inducing the strong psychoactive effects of known from classical psychedelics or entactogens. As such, they can also cause neuroplasticity with or without being psychoactive or at doses that are only moderately psychoactive or the substances increase levels of monoamines such as dopamine, norepinephrine or serotonin or oxytocin or BDNF or induce neuroplasticity or markers thereof. Increase of monoamines can, for example, be a result of release, uptake inhibition, amine oxidase inhibition or interaction with the vesicular monoamine transporters similar to anti-Parkinsonian and antidepressant agents such as rasagiline, selegiline, both containing a propargylamine function, as well as of amphetamine, used e.g., for ADHD treatment, and many structurally related compounds thereof.

The assumption of comparable pharmacological properties of the compounds of invention to phenethylamine- or tryptamine-based psychedelics or entactogens is further emphasized by structure-activity relationships. Although with limited data available, psychedelic phenethylamines extended from an α-methyl to an α-ethyl group can either be acutely psychoactive (e.g., the DOB homologue 4C-B, a mixed psychedelic-energizing active compound in the range of 50-80 mg (Trachsel, Lehmann, & Enzensperger, 2013) or act only as nootropics with no psychedelic effects, e.g. 4C-D, also known as Ariadne, for which possible mechanistic explanations have been given (Cunningham et al., 2023). The potential for therapeutical use of Ariadne has been investigated clinically, and results were very promising in treatments such as rapid remission of psychotic symptoms in schizophrenics, relaxation in catatonics, complete remission of symptoms in Parkinson's disease (PD), and improved cognition in geriatric subjects (summarized in (Cunningham et al., 2023)). Mechanistically, Ariadne and related compounds still behave as 5-HT2A receptor agonists, which is the primary target for psychedelic compounds, including many phenethylamines, tryptamines and ergolines. However, Ariadne and related compounds have shown to be compounds with significantly lower signaling potency and efficacy in different signaling pathways (Gq, G11, and β-arrestin2) coupled to 5-HT2A receptors in comparison to the prototypical psychedelic phenethylamine DOM (2,5-dimethoxy-4-methylamphetamine), which can explain the lack pf psychedelic effects (called as signaling efficacy hypothesis by the authors of the study (Cunningham et al., 2023)). Depending on the aryl substituents introduced into the phenethylamines, stimulants, entactogens or psychedelics can be obtained (for extensive structure-activity relationships details consult e.g., (Trachsel et al., 2013). As outlined above, an extension of an α-methyl to an α-ethyl group within the phenethylamine-type psychedelics leads to a dramatical loss of potency. This contrasts with what has been observed among some entactogenic phenethylamines, e.g., the same α-ethyl extension largely retains potency and psychoactive effects, with some differences in quality of experienced effects. To name are exemplarily the comparators MDMA vs. MBDB and MDA vs. BDB (A. Shulgin & Shulgin, 1991). For tryptamines, it has been shown that introduction of an α-methyl group can have dramatic influence on potency and psycho activity (A. T. Shulgin & Shulgin, 1997). As such, while unsubstituted tryptamine is completely inactive, α-methyl-tryptamine is a relatively potent psychedelic with some significant side effects probably also due to its monoamine oxidase inhibiting abilities (Arai, Toyoshima, & Kinemuchi, 1986); at much lower doses it formerly was used as an antidepressant (Iversen, 2013). This compound behaves as non-selective 5-HT receptor ligand and as monoamine transporter agent (5-HT, NE and DA) (Nagai, Nonaka, & Satoh Hisashi Kamimura, 2007; Nonaka, Nagai, Ogata, & Satoh, 2007). Extension to an α-ethyl group leads to α-ethyl-tryptamine, a compound with psychoactive properties more similar to the entactogenic MDMA than to psychedelics. α-Ethyl-tryptamine also has monoamine oxidase inhibiting properties and was investigated as an antidepressant but was withdrawn from potential commercial use due to incidences of agranulocytosis (A. Shulgin & Shulgin, 1991). Its primary pharmacological principle is thought to be the release of serotonin, norepinephrine and dopamine similar to MDMA (Blough et al., 2014). In summary, among the tryptamines, introduction of α-substituents, at least up to an ethyl group, are tolerated as well, leading to significantly altered pharmacological properties.

With the invented dehydrated compounds of FIGS. 1B-C, leading to allyl- or propargylamine-type phenethylamines or tryptamines or similar compounds, the compounds are sterically comparable to the corresponding α-ethyl derivatives with having a similar or slight smaller molar refraction. However, physico-chemical properties such as hydrophobicity, Hammett constant and electronegativity of an ethenyl or ethynyl group significantly differ from an ethyl group, allowing to potentially influence the overall properties of compounds of invention.

With the invented β-unsaturated phenethylamines, tryptamines, and other aryl- or heteroarylethylamines, a second type of allylamines are obtained (FIG. 1A). Physico-chemically, these β-methylidene compounds differ from the well-known β-keto phenethylamines such as methylone or butylone, a group that also represents compounds with some entactogenic and stimulant properties. For methylone, e.g., a comparable or slightly reduced potency and a similar duration of action is known in comparison to MDMA, although the entactogenic properties are diminished (Simmler et al., 2013). From these as well as from other compounds, e.g., β-methylated or β-methoxylated phenethylamines, it is known that β-substitution is tolerated up to a certain extent and can retain some of the pharmacological activities (A. Shulgin & Shulgin, 1991; Trachsel et al., 2013). Among the β-methylidene derivatives, one α-methyl substituted as well as a few α-unsubstituted compounds have been investigated as bovine chromaffin vesicular monoamine transporter Inhibitors agents (Perera, Wimalasena, & Wimalasena, 2003), but none contained substitution patterns as defined for FIGS. 1A-E.

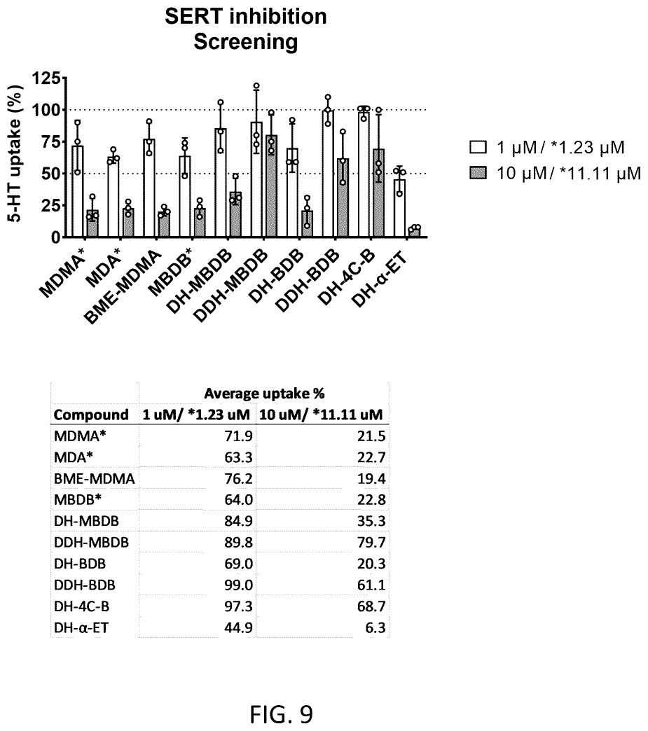

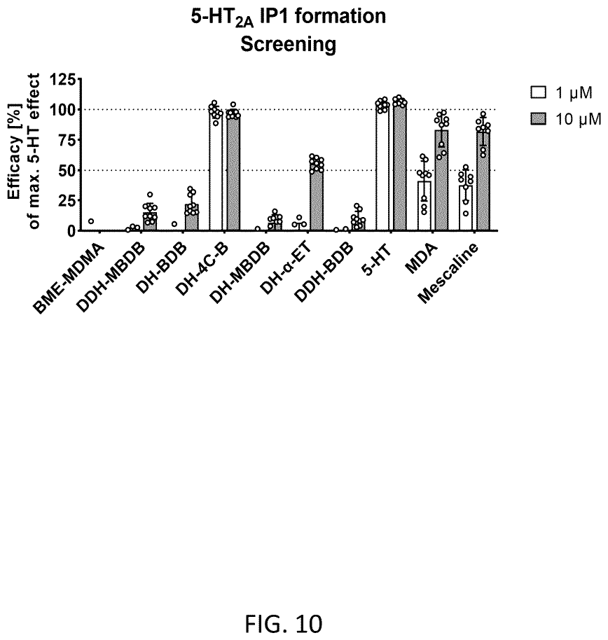

The present invention provides compounds of FIGS. 1A-1E that are pharmacologically active and allow changing the neurotransmission and/or producing neuroplasticity. More specifically, but not exclusively, the compounds interact with serotonin (5-HT, 5-hydroxytryptamine) 5-HT2A receptors and with monoamine transporters and/or with amine oxidases (FIG. 9 and FIG. 10) in mammals by administering to a mammal in need of such interaction a pharmaceutically effective amount of a compound of FIGS. 1A-1E.

Therefore, the present invention provides a method of changing neurotransmission, by administering a pharmaceutically effective amount of a compound of FIGS. 1A-1E to a mammal, increasing serotonin 5-HT2A receptor interaction and/or monoamine levels in the mammal, and inducing psychoactive effects.

The neuronal effects of compounds represented in FIGS. 1A-1E can be used in mammals for treating medical disorders and substance-assisted therapy where the compounds induce psychoactive effect, for example to enhance psychotherapy. The preferred mammal is human.

Further on, the neuronal effects of compounds represented in FIGS. 1A-1E can also be used in mammals for pro-cognitive effects or for therapy or psychotherapy where the compounds induce less dominant or no acute psychoactive effect. The preferred mammal is also human.

Specifically, the compounds can be used in compound-assisted therapy for medical disorders including post-traumatic stress disorder, social anxiety, autism spectrum disorder, substance use disorder (including for alcohol, stimulants, or opioids), depression (including major depression disorder), psychotic symptoms, relief from symptoms of Parkinson's disease, cognition disorders, anxiety disorder (including generalized anxiety disorder), anxiety with life-threatening disease, personality disorder including narcistic or antisocial personality disorder, obsessive compulsive disorder, attention-deficit/hyperactive disorder, eating disorder (anorexia and bulimia), pain (including cluster headache, migraine, fibromyalgia, neuropathic pain, cancer pain, and low back pain), couple therapy, enhancement of any psychotherapy by inducing feelings of well-being, connectedness, trust, love, empathy, openness, and pro-sociality, and enhancing therapeutic alliance in any psychotherapy of patients or neurotic/healthy subjects.

Based on the pharmacological data generated and shown (FIG. 9 and FIG. 10) and on structure-activity relationships the invented derivatives and related compounds presented in FIGS. 1A-1E behave either as 5-HT2A receptor ligands and are therefore like psychedelics, and/or as monoamine transporter interaction agents or as amine oxidase inhibitors, or any combination thereof, and act therefore like entactogens and/or are e less or not acutely psychoactive with potential enhancing cognitive and emotional positive effects in treating mental disorders.

The structural modifications presented in FIGS. 1A-1E may also alter absorption, distribution, metabolism and excretion (ADME) properties compared to existing treatments, e.g., the metabolism can be modified significantly by making, as an example, but not limited to, a potentially labile alkenyl or alkynyl compound more or less prone to metabolism by introducing alkyl groups, aryl or heteroaryl groups, nitriles, fluorine atoms and deuterium atoms to these functional groups in either vinyl, allyl or gamma positions, or in ethynyl or propargyl positions. Thus, the invention allows also for the synthesis of compounds with a relatively shorter duration of action compared to more metabolically stable and longer-acting existing compounds.

While all the phenethylamine and tryptamine derivatives and related compounds represented in FIGS. 1A-1E are useful in optimizing the clinical effect profile of psychedelics, entactogens, pro-cognitive and mood disorder-treating agents, certain classes/forms of the compounds are preferred, such as wherein the compound is a free base, a salt, a hydrochloride salt, a fumarate salt, a hemifumarate salt, a racemate where applicable, a single enantiomer, a single diastereomer, or a mixture of enantiomers or diastereomers in any ratio, or an individual of a cis/trans or EIZ configurational isomer, or a mixture of these configurational isomers in any ratio. It will be understood that these classes/forms can be combined to form additional preferred classes.

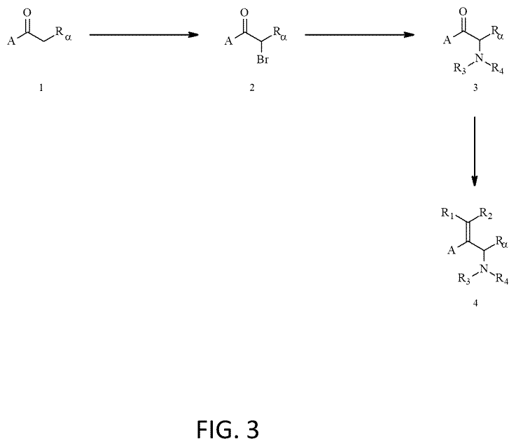

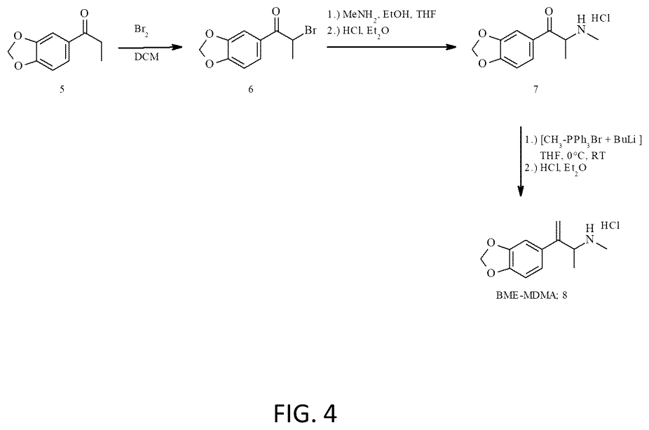

The synthetic access to β-alkylidene derivatives (allylamines) such as represented in FIG. 1A is outlined generically in FIG. 3 and, for the representative compound 8, in FIG. 4. The introduction of an alkene moiety in a benzyl position has been described and reviewed briefly by Perera et al. (Perera et al., 2003), but none of the compounds therein contained substitution patterns as defined for FIGS. 1A-E. Namely, a Grignard reaction onto acetophenones and subsequent acetylation/dehydration with acetic anhydride lead to the α-methylstyrenes, and an allylic bromination with N-bromosuccinimide (NBS), followed by a Gabriel phthalimide synthesis lead to their β-methylidene derivatives, and alternatively, they could be accessed via direct Wittig reaction with acetophenones. However, with an electron-rich group attached to the phenyl ring, namely a methoxy substituent, the introduction of a bromine in allylic position failed, and vinylic bromination was the exclusive reaction observed. Thus, the inventors chose a different approach to access compounds represented in FIG. 1A. It can be followed the general access to the known β-keto substituted phenethylamines, namely, a correspondingly aryl- or heteroaryl substituted acyl derivative 1 is α-brominated by conditions such as bromine in dichloromethane (DCM) or other solvents, or with CuBr₂ in a suitable solvent such as EtOAc or DCM, then a dehalo-amination of 2 with a correspondingly substituted amine in a suitable solvent such as an alcohol such as methanol, ethanol or isopropanol or an ether such as dioxane or tetrahydrofuran (THF) is performed to access a β-keto amine 3 (FIG. 3). The amine to be used for this reaction can be a free base, a salt or accordingly protected. In case an amine salt is used, a suitable organic or inorganic base like triethylamine or NaOH is added to the reaction. The β-keto amine 3 can be isolated as free base, as a salt or as an N-protected amine and is finally converted directly to the β-methylidene derivative 4 by using a Wittig type reaction by using Wittig ylides generated in situ from a Wittig salt or Wittig type reagent and a base such as BuLi, LHMDS or LDA, in a suitable solvent such as THF, DMSO or the like, at a temperature like −100 to 70° C., more preferably at −80 to 30° C. Depending on the reaction conditions and reagents and additives used, one can force the reaction to yield a preferred amount of either of the configurational alkene isomers E or Z or cis or trans, where applicable. Alternatively, the configurational isomers can also be separated by any suitable technique such as chromatography, crystallization, or distillation. In case of using an amine salt 3, the β-methylidene derivatives 4 can be isolated as free base and converted to a suitable salt such as a hydrochloride. When using an N-protected β-keto amine 3, the corresponding β-methylidene intermediate obtained from the Wittig reaction can be N-deprotected and then be isolated as free base or as an amine salt such as a hydrochloride. As a representative example, FIG. 4 outlines such a synthetic procedure to access the example compound 8, a compound represented by FIG. 1A. This order of reaction sequence has the advantage of late-stage diversification around the double bond to be introduced, and many different Wittig reagents can be used. Further on, as shown by the inventors, the Wittig reaction can be performed onto the β-keto amines without the use of N-protection groups. Applying Wittig reactions onto α-bromoketones, e.g. onto structure 2 leads either to complex reaction mixtures or to stable oxaphosphetane intermediates (unpublished findings), and also alternative conditions such as the use of CH2Br2, TiCl4 and Zn (Kazuhiko, Yuji, Koichiro, & Hitosi, 1980) lead to complex reaction mixtures.

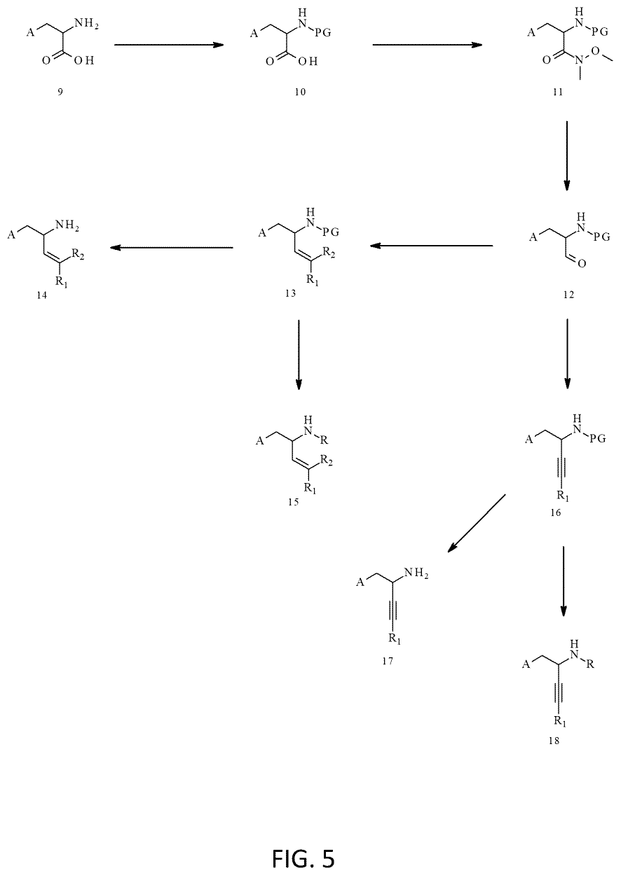

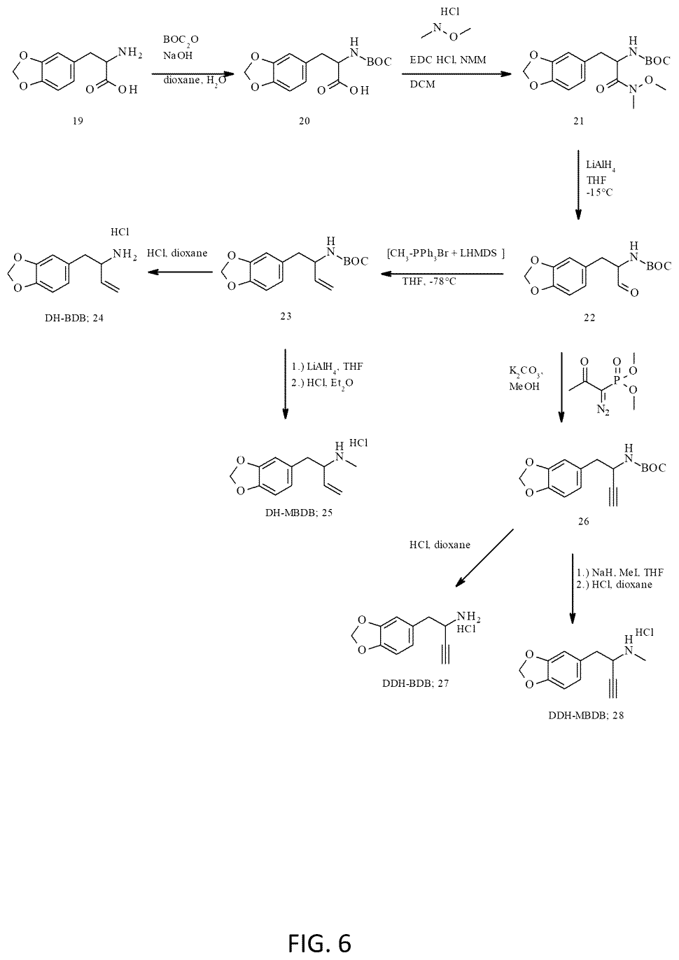

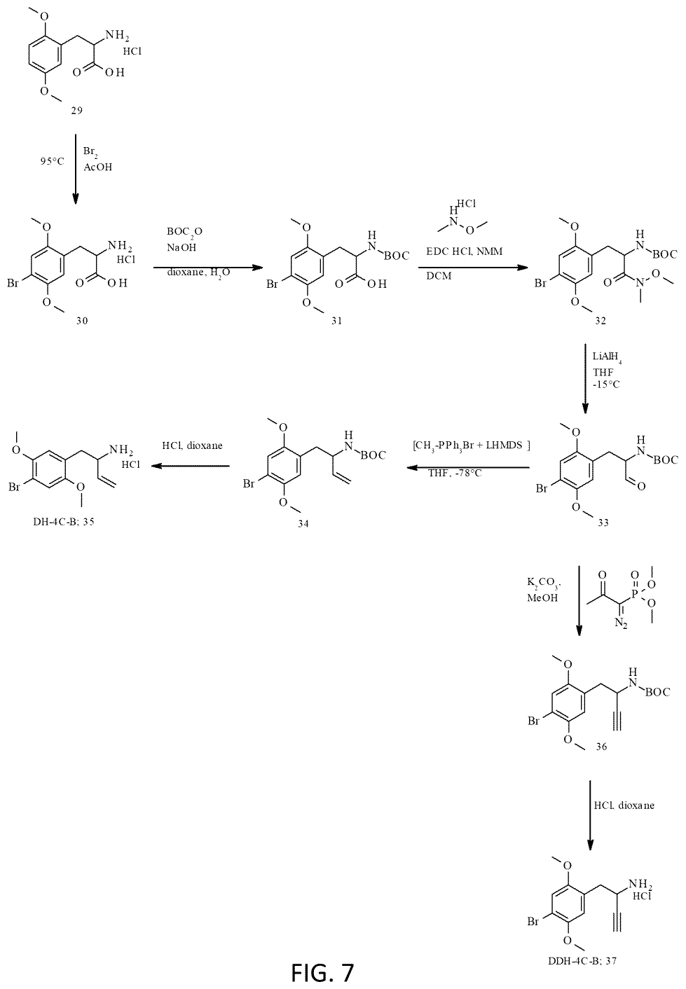

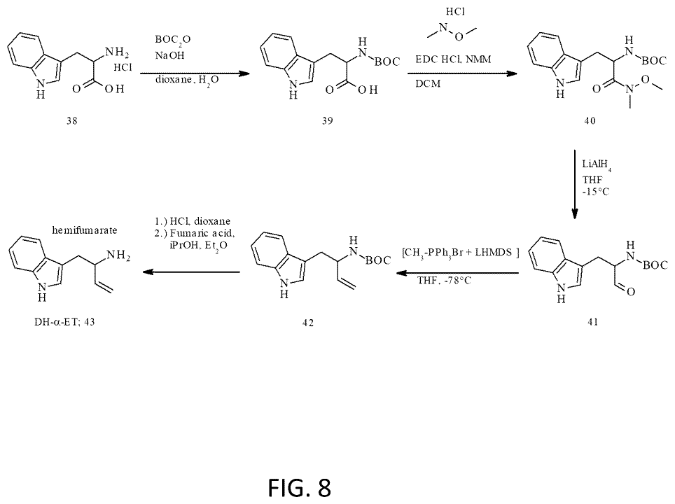

Accessing unsaturated derivatives such as alkenyl derivatives (allylamines) represented in FIG. 1B is outlined generically in FIG. 5 and, for the representative compounds 24, 25, 35, 41 and 43, in FIGS. 6-8. It can be followed on known routes such as used by Blacker et al. (Blacker et al., 2011) or, more suitably for compounds of invention, the route described by Velmourougane et al. (Velmourougane et al., 2011) and Janssen et al. (Janssen et al., 2019). As such, a correspondingly 3-aryl- or 3-heteroaryl substituted 2-aminopropionic acid (alanine) 9 is N-protected with a suitable protecting group like tert-butoxycarbonyl (BOC), the obtained intermediate 10 is then converted to a Weinreb amide such as compound 11, and the Weinreb amide can be reduced to the corresponding aldehyde 12 (FIG. 5). Aldehyde 12 can be converted to the corresponding alkene 23 by applying a Wittig or Wittig type reaction by using Wittig ylides generated in situ from a Wittig salt or Wittig type reagent and a base such as BuLi, LHMDS or LDA, in a suitable solvent such as THF, DMSO or the like, at a temperature like −100° C. to 70° C., more preferably at −80° C. to 30° C. Depending on the reaction conditions and reagents and additives used, one can force the reaction to yield a preferred amount of either of the configurational alkene isomers E or Z or cis or trans, where applicable. Alternatively, the configurational isomers can also be separated by any suitable technique such as chromatography, crystallization, or distillation. The N-protected alkenyl derivative 13 can be N-deprotected to the allylamine 14 and then be isolated as free base or as an amine salt such as a hydrochloride or a fumaric acid salt. The allylamine 14 can also be further derivatized on the basic nitrogen to access further examples such as structure 15 and represented with FIG. 1B. The N-protected alkenyl derivative 13 can also be used directly to obtain N-substituted examples (structure 15) represented by FIG. 1B. As such, when 13 is containing an N-BOC group, it can be N-alkylated or directly reduced to an N-methyl-containing compound. As representative examples, FIG. 6 outlines such synthetic procedures to access the example compounds 24 and 25, compounds represented by FIG. 1B, and FIGS. 7 and 8 outline such synthetic procedures to access the example compounds 35 and 43, respectively, compounds also represented by FIG. 1B.

Accessing unsaturated derivatives such as alkynyl derivatives (propargylamines) as represented in FIG. 1C is outlined generically in FIG. 5 and, for the representative compounds 27 and 28, in FIG. 6, and, for the representative compound 37, in FIG. 7. The authors Roth, et al. presented a general access to alkynes starting from aldehydes by using the reagent dimethyl-(1-diazo-2-oxopropyl) phosphonate (Roth, Liepold, Mueller, & Bestmann, 2004). With this, the above aldehyde intermediate 12 (FIG. 5) can easily be converted to the alkyne compound 16 in the presence of a base like K2CO3 in a solvent like methanol. Other conditions can be used as well, and transformations are not limited to this specific reagent. The N-protected intermediate 16 can then be deprotected to access propargylamine 17 which can be isolated as free base or as a salt such as a hydrochloride or a fumaric acid salt. Further on, intermediate 16 can also be converted to an N-alkylated compound 18 by applying suitable alkylating conditions such as a strong base like NaH and an alkylating agent like an alkyl halide, and by subsequent N-deprotection with, in case of an N-BOC protecting group, an acid such as HCl in a solvent like dioxane. Intermediate 16 can also be reduced directly to an N-alkyl derivative 18, as an example when using N-BOC as a protecting group in 16, reduction with a reducing agent like LiAlH4 in a solvent like THE leads to an N-methyl compound 18. As representative examples, FIG. 6 outlines such synthetic procedures to access the example compounds 27 and 28, compounds that are represented by FIG. 1C.

Accessing β,β′-unsaturated derivatives such as dialkenyl derivatives represented in FIG. 1D and such as alkenylalkynyl derivatives represented in FIG. 1E can be accessed by combination of some of the above reactions in a suitable order, or by using additional or different chemical transformations. Suitable starting materials can be but are not limited to β-aryl-substituted β-keto-amino acids.

The group presented in the preparation section, namely compounds 8, 24, 25, 27, 28, 35, 41 and 43 (chemical structures see FIGS. 2A-2H), is illustrative of allyl- or propargylamine-type phenethylamines and tryptamines and related compounds represented in FIGS. 1A-1E contemplated within the scope of the invention.

A small selection of the allyl- or propargylamine-type phenethylamines and tryptamines and related compounds as shown in FIGS. 1A-1E were investigated at the key targets for psychoactive effects in vitro (FIG. 9 and FIG. 10). The main target of psychedelics is the 5-HT2A receptor (Becker et al., 2022; Holze, Vizeli, et al., 2021) and typically there is a high affinity binding at this receptor (Rickli et al., 2016). Additionally, the binding potency at the 5-HT2A receptor is typically predictive of the human doses of psychedelics to be psychoactive for many compounds (Luethi & Liechti, 2018). Furthermore, the psychedelic effects of psilocybin in humans have been shown to correlate with 5-HT2A receptor occupancy measures using positron emission tomography (Madsen et al., 2019). Thus, interactions with this target are relevant and predict psychedelic action with high likelihood for most psychedelics. However, this may not be the case for all substances within this class, and less acute psychoactive effects can be obtained, due to significantly lower signaling potency and efficacy in the downstream signaling pathways (Gq, G11, and β-arrestin2) coupled to 5-HT2A receptors (Cunningham et al., 2023) or by biased signaling wherein one pathway is less active relative to another for one substance relative to another. Compounds with such properties can be useful as cognitive or mood enhancers or in some forms of substance-assisted therapy where less or no acute psychoactive effect is preferred.

For some of the compounds invented, activity at the 5-HT2A receptor could be presented (FIG. 10), and for the invented compounds DH-4C-B (22.1 nM) and DDH-4C-B (40.7nM) were found IC50 values at the 5-HT2A receptor showing significant interactions (values for hydrochloride salts).

Additional receptors such as the serotonergic 5-HT1A and 5-HT2C or dopaminergic D2 receptors are thought to moderate aspects of the effects of psychedelics (Rickli et al., 2016). Although some psychedelics like psilocybin do not directly act on dopaminergic receptors, they have nevertheless some dopaminergic properties by releasing dopamine in the striatum (Vollenweider, Vontobel, Hell, & Leenders, 1999) likely via 5-HT1A receptor activation (Ichikawa & Meltzer, 2000). Furthermore, LSD has activity at D2 receptors (Rickli et al., 2016) and some of its behavioral effect can be linked to this target (Marona-Lewicka, Thisted, & Nichols, 2005).

Activity of compounds at monoamine transporters are thought to mediate MDMA-like entactogenic effects (Hysek et al., 2012). Entactogens exert their pharmacological action primarily by an acute release and reuptake inhibition of monoamines with a preference for serotonin over dopamine (M. Liechti, 2015; Simmler et al., 2013) and the window leading to entactogenic effects versus pure stimulant effects seems to be narrow. Numerous compounds were investigated (e.g., MDA, MDEA, MBDB, the benzofurans 5-APB, 6-APB, or the aminoindane MDAI), however, the unique effects of MDMA including facilitated communication, insight, trust, and self-acceptance seems not fully reached using these compounds (A. Shulgin & Shulgin, 1991).

The test substances were studied at 1μM and 10μM concentrations (n=3). Concentrations >10 μM were considered not pharmacologically relevant. The efficacy of the test substances at the 5-HT_{2A} receptor, expressed as a percentage of the maximal 5-HT response, is shown in FIG. 10. 3,4-Methylene-Dioxy-Amphetamine (MDA) and mescaline were included as reference compounds.

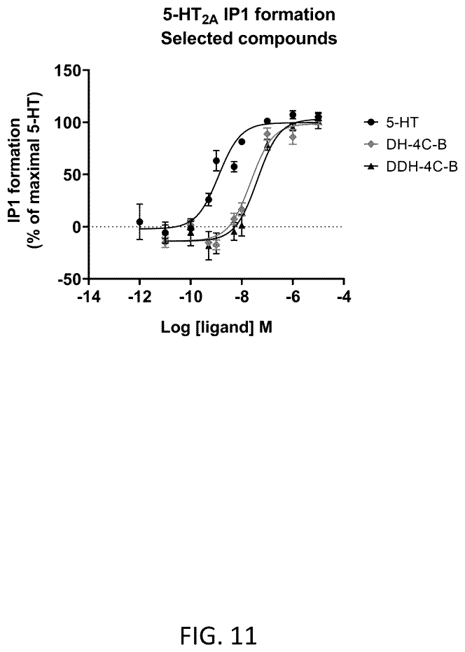

Based on the obtained efficacy data shown in FIG. 10, DH-4C-B and DDH-4C-B were selected for detailed 5-HT_{2A}-receptor activation assessment. DH-4C-B and DDH-4C-B fully activated the receptor with EC₅₀ values of 22.14 nM and 40.67 nM, respectively, as shown in FIG. 11. For the sake of completeness, the comparison also included natural substrate 5-HT that activated the receptor with an EC₅₀ of 1.2 nM.

Only DH-α-ET displayed a sub-micromolar IC₅₀ value. BME-MDMA, DH-BDB, and DH-MBDB showed an IC₅₀ value in the range of 1-10 μM, whereas DDH-MBDB, DH-4C-B, and DDH-BDB showed an IC₅₀ value >10 μM. Thus, for some compounds of the invention (BME-MDMA, DH-BDB, and DH-MBDB) a similar or even greater inhibition of the SERT was observed as for MDMA or MDA (Simmler et al., 2013 and data on file) (FIG. 9). MDMA exhibits an IC₅₀ value of approximately 1.4 μM in this assay (Simmler et al., 2013).

In conclusion, the biological screening data performed for this invention indicate that BME-MDMA, DH-BDB, and DH-MBDB inhibited SERT with similar potency as MDMA and MDA (IC₅₀=1−10 μM). However, all three substances displayed weaker activation efficacy at the 5-HT_{2A} receptor when compared to MDA. DH-4C-B showed potent activity at the 5-HT_{2A} receptor but lacked relevant SERT inhibition potency (IC₅₀>10 μM). DH-α-ET was a potent inhibitor of 5-HT reuptake (IC₅₀<1 μM) as well as a moderately potent activator at the 5-HT_{2A} receptor (˜50% efficacy at 10 μM). Taken together, this pharmacological screen shows that DH-α-ET, BME-MDMA, DH-BDB, and DH-MBDB likely act as entactogens, with DH-α-ET possibly mediating some slight psychedelic-like effects. The profiles of DH-4C-B and DDH-4C-B shows them to be psychedelics rather than entactogens. However, it is important to note that in addition to 5-HT_{2A} receptor and SERT interactions, several other properties, for instance, interactions with other monoamine transporters and receptors, can influence the distinct pharmacological profile of the test substances and ADME properties also influence the clinical potency and efficacy of these compounds.

Inhibiting amine oxidases can also lead to increased monoamine levels, representing another concept of treating monoamine-related mental illness such as mood disorders. Phenethylamines and tryptamines of different structures have been shown to behave as monoamine oxidase inhibitors, and several compounds have been evaluated clinically or are currently used to treat depression or Parkinson's disease. Compounds such as rasagiline or selegiline, which both bear a propargylamine function, are effective therapeutics as such. Compounds represented by FIGS. 1A-E share some structural features with known monoamine oxidase inhibitors and can bear, additionally or aside to psychedelic or to entactogenic properties, amine oxidase inhibiting properties, allowing them also to be used for treatments where less or no acute psychoactive effect will be needed, such as in mood disorders or pro-cognitive needs.

The serotonin 5-HT2A receptor activation and serotonin transporter inhibition data (FIG. 9 and FIG. 10) suggests that the compounds from FIGS. 1A-1E are active as psychedelics and/or entactogens since they bear structural features of known psychedelics and/or entactogens and are active at these targets and to be evaluated clinically. Key results of the preliminary pharmacological profiling of the compounds described herein were:

Some of the phenethylamines, tryptamines and related derivatives containing an allyl- and/or propargylamine moiety represented in FIGS. 1A-1E showed activity at the serotonin 5-HT2A receptor and/or inhibiting properties at the serotonin transporter indicating activity as psychedelics and/or entactogens.

Together, the in vitro profiles of phenethylamines, tryptamines and related derivatives containing an allyl- and/or propargylamine moiety represented in FIGS. 1A-1E compared with that of psilocybin, LSD and MDMA indicate overall psychedelic and/or entactogenic properties when used in humans. Accordingly, some phenethylamines, tryptamines and related derivatives containing an allyl- and/or propargylamine moiety can exert psychedelic and/or entactogenic acute effect profiles that are more beneficial to some patients including but not limited to: more overall positive effects, more or less perceptual effects, more emotional effects, less anxiety, less cardio-stimulant effects, less adverse effects, less nausea, longer and also shorter effects among other properties and compared to compounds such as psilocybin, LSD or MDMA.

There are several problems when using known psychedelics such as LSD that can be solved using the compounds described herein. Namely, long therapeutic session durations (LSD: 8-12 hours, DOM 14-20 hours) are needed due to the long duration of psychoactive effects, and thus either therapists need to care their patients for an unreasonable long time—with that being significant economic disadvantages—, or psychedelic trips need to be stopped by administering an antidote such as a serotonin 5-HT2 antagonist (U.S. Ser. No. 17/156,233) or a benzodiazepine, which in certain cases can be disadvantageous. The presently developed substances represented in FIGS. 1A-1E were also designed to have similar qualitative effects to phenethylamines such as DOM or MDMA while acting shorter or to have a comparable duration of action but other qualitative effects as reflected by their structural changes and associated pharmacological properties. Other alterations of the chemical structure were designed to create substances with qualitative effects different from those of known psychedelics or entactogens, and creating subjective effects that are considered beneficial to assist psychotherapy including feelings of empathy, openness, trust, insight, and connectedness and known to those knowledgeable in the field. Further problems when using known psychedelics like psilocybin can rise from producing adverse effects including nausea and vomiting, cardiovascular stimulation, and an increase in body temperature and others. The novel compounds can produce less nausea, less cardio stimulation, less thermogenesis and/or other adverse responses.

The compounds represented by FIGS. 1A-1E act with shorter or with comparable duration of action in human in comparison to such phenethylamines such as the DOM or MDMA molecule. This is triggered by modification of the molecular structure in FIGS. 1A-1E.

The present invention therefore also provides for a method of treating a patient having adverse reactions to psychedelics by administering a phenethylamines, tryptamines and related derivatives containing an allyl- and/or propargylamine moiety to the patient and avoiding adverse effects present with psychedelics.

For entactogens, only MDMA is clinically evaluated (Mitchell et al., 2021) for its use as a therapeutic tool. For MDMA, certain disadvantages are known, such as an overwhelming onset, and adverse effects that make MDMA not a suitable therapy tool for some patients. Alternative compounds with different pharmacological profiles are thus needed creating subjective effects that are considered beneficial to assist psychotherapy including feelings of empathy, openness, trust, insight, and connectedness and known to those knowledgeable in the field. MDMA can produce adverse effects including nausea and vomiting, cardiovascular stimulation, bruxism, dehydration and an increase in body temperature and others. The novel compounds represented in FIGS. 1A-F can produce less nausea, less cardio stimulation, less thermogenesis and/or other adverse responses.

The group presented in the preparation section, namely compounds 8, 24, 25, 27, 28, 35, 41 and 43 (chemical structures see FIGS. 2A-2H), is illustrative of phenethylamines, tryptamines and related derivatives containing an allyl- and/or propargylamine moiety represented in FIGS. 1A-1E contemplated within the scope of the invention.

The compounds according to the invention and represented in FIGS. 1A-1E allow modification of the duration and mode of action, the psychodynamic processes, and the qualitative perceptions, e.g., in terms of psychedelic or entactogenic intensity in comparison to phenethylamines such as DOM or MDMA.

The compounds according to the invention and represented in FIGS. 1A-1E can cause similar or different quality of imagery, fantasy and closed or open eyes visuals in comparison to phenethylamines such as DOM.

The compounds according to the invention and represented in FIGS. 1A-1E can cause similar or different quality of feelings of well-being, feelings of connectivity to others, feelings of increased trust, feelings of love, enhanced emotional empathy, and enhanced feelings of pro-sociality and prosocial behavior in comparison to MDMA.

The compounds according to the invention and represented in FIGS. 1A-1E can have a lower, a similar or a higher dose potency in comparison to phenethylamines such as DOM or MDMA.

The compounds according to the invention and represented in FIGS. 1A-1E can cause similar or more favorable body feelings in comparison to phenethylamines such as DOM or MDMA.

The aforementioned characteristics can be additionally modified in a progressive way by the introduction of one or more fluorine atoms, by one or more deuterium atoms and by one or more alkyl groups, independently as well as in any combination, to the alkenyl or alkynyl chain in any position of these groups or substituents.

The aforementioned characteristics can further be modified in a progressive way by the introduction of one or more fluorine atoms, by one or more deuterium atoms, by one or more alkyl group, and by one or more functional group such as carbon-, nitrogen-, oxygen-, or sulfur-containing, independently as well as in any combination, to any position of the compounds represented in FIGS. 1A-1E, namely to the aryl- or heteroaryl substituents, to the aromatic or heteroaromatic nucleus, to the alkylamine side chain, to the nitrogen substituents or to any other introduced substituent on the compounds of invention.

This is outlined in more detail by the following relationships. The overall chemical/biological stability of a compound of invention as represented in FIGS. 1A-1E towards acidic, basic or any other chemical/biological conditions is dependent on factors such as pH, solvent medium, temperature, surface, nucleophilicity or electrophilicity of reaction partners or on gas containment of the environment. Metabolic stability in a biological environment such as a human body is additionally driven by factors such absorption rate, exposure to enzymes, enzyme activity, genetic polymorphism, retention time in a body medium such as gastrointestinal tract, rate of body distribution or transportation times. All these aspects can be influenced by the structural changes introduced to the compounds and result in the desired effects and effect-durations in humans.

The stability of a functional group, a substituent or, generally spoken, a molecule, towards aforementioned factors can significantly be influenced and modified by specific incorporation of stabilizing or destabilizing atoms or atom groups. Furthermore, the overall metabolic stability of a compound is also driven by properties such as the overall lipophilicity, three-dimensional structure, dissociation constants, solubility, steric accessibilities and steric bulkiness and other characteristics.

Fluorine is a strong electron-withdrawing atom and its incorporation to a substituent can significantly reduce the electron richness. Further on, it modifies dipole moment, dissociation constants of acidic and basic groups, the lipophilicity, pH value, and, to a certain extent, also steric properties of a fluorine-containing molecule are influenced. Thus, fluorine can chance physicochemical properties and incorporation into a molecule can have a dramatic influence on interaction with biological targets, on chemical/metabolic stabilities and on metabolic pathways. Fluorine atoms incorporated to a molecule further allows so-called multipolar interactions with partially charged functional groups. This makes fluorine as an excellent tool for medicinal chemistry.

Deuteron is a stable isotope of hydrogen. Due to its slightly different metric, incorporated into a molecule it can influence physicochemical properties. With this, kinetic isotope effects, inverse kinetic isotope effects and steric isotope effects can also be observed. Chemical bonds involving deuterium are stronger and of different length compared to protium (hydrogen), which make such compounds significantly different in biological reactions. Thus, incorporation of a deuteron into a molecule can greatly influence its biological stabilities.

Consequently, both fluorine and deuteron can be used to replace or to be added to a substituent to modify the overall stability and biological properties of the compounds invented and represented by FIGS. 1A-1E. Replacement can be done with one or more fluorine atoms or with one or more deuteron atoms or in any combination of fluorine and deuteron atoms.

In analogy to these medicinal chemistry concepts, the biological properties of the invented compounds (ADME, target selectivity and target interaction, the mode of action, duration of action, the psychodynamic processes, and the qualitative perceptions, e.g., in terms of psychedelic or entactogenic intensity) can not only be influenced by the aforementioned application of fluorine or deuteron to the functional groups such as the alkenyl or alkynyl chain but also to any position of the structure of the compounds in FIGS. 1A-1E. Other atoms or atom groups can be used in a similar way to all these positions.

Taken together, the modified properties can be tailored and applied individually to the patient's need. This is not only targeted by changing the compound's receptor profile but also greatly by the modification of ADME (Absorption, Distribution, Metabolism and Excretion) via the scope of invention outlined before in compounds represented in FIGS. 1A-1E.

The compound of the present invention is administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, or parenterally including intravenous, intraarterial, intramuscular, intraperitoneally, subcutaneously, and intranasal administration as well as intrathecal and infusion techniques. Implants of the compounds are also useful. The patient being treated is a warm-blooded animal and, in particular mammals, including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, using a coating such as lecithin, by the maintenance of the required particle size in the case of dispersion and using surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about using agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather should be construed to encompass any and all variations which become evident as a result of the teaching provided herein.

### Example 1

### Biological Evaluation of the Compounds

Serotonin uptake transport inhibition. Inhibition of the human serotonin (5-HT) transporter (hSERT) was assessed in stably transfected human embryonic kidney (HEK) 293 cells (Invitrogen, Zug, Switzerland). Briefly, cells were cultured in Dulbecco's modified Eagle's medium (DMEM; Gibco, Life Technologies, Zug, Switzerland) with 10% fetal bovine serum (Gibco) and 250 mg/mL Geneticin (Gibco) to 70-90% confluence, detached, and then resuspended (3×10⁶ cells/mL) in Krebs-Ringer Bicarbonate Buffer (Sigma-Aldrich, Buchs, Switzerland). The cell suspension (100 μL) was incubated with 25 μL buffer containing the test compounds, vehicle control, or 10 μM fluoxetine (SERT-specific inhibitor) for 10 minutes in a round bottom 96-well plate at room temperature by shaking at 450 rotations per minute on a rotary shaker. To initiate uptake transport, 50 μL of [³H] 5-HT dissolved in uptake buffer was added at a final concentration of 5 nM for an additional 10 minutes. Thereafter, 100 μL of the cell suspension was transferred into 500 μL microcentrifuge tubes that contained 50 μL of 3 M KOH and 200 μL silicon oil (1:1 mixture of silicon oil types AR 20 and AR 200; Sigma-Aldrich). The tubes were centrifuged for 3 minutes at 16,550 g to transport the cells through the silicone oil into the KOH. The tubes were frozen in liquid nitrogen and the cell pellet was then cut into 6 mL scintillation vials (PerkinElmer, Schwerzenbach, Switzerland) that contained 0.5 mL lysis buffer (0.05 M TRIS-HCl, 50 mM NaCl, 5 mM EDTA, and 1% NP-40 in water). The samples were shaken for 1 hour before the addition of 5 mL scintillation liquid (Ultimagold, Perkin Elmer). Serotonin uptake was quantified by liquid scintillation counting on a Packard Tri-Carb Liquid Scintillation Counter 1900 TR. Non-specific uptake in the presence of fluoxetine was subtracted from the total counts.

5-HT_{2A} receptor activation assessment. Activation of the 5-HT_{2A} receptor was assessed by measuring the accumulation of inositol monophosphate 1 (IP1) using the Cisbio IP-One Gq Kit (Cisbio Bioassays SAS, Codolet Cedex, France) according to the manufacturer's protocol. In brief, NIH/3T3 cells stably expressing the human 5-HT_{2A} receptor were seeded at a density of 2,500 cells per well in a 384-well plate in Opti-MEM™ I medium (Gibco, ThermoFisher, Life Technologies Europe B.V., Zug, Switzerland). Then, test compounds were added, and the plate was incubated for 90 minutes at 37° C., followed by 60 minutes incubation with Anti-IP1-Cryptate and IP1-d2 at room temperature. Stimulated IP-1 formation was determined by Homogeneous Time-Resolved Fluorescence (HTRF) measurement on a BioTek Synergy H1 Multimode Reader (Agilent Technologies (Schweiz) AG, Basel, Switzerland) and EC₅₀ values were derived from the concentration-response curves using nonlinear regression.

### Example 2

### Preparation of the Compounds

A general access to the allyl- or propargylamine-type phenethylamines and tryptamines and related compounds as shown in FIGS. 1A-1E is outlined in FIGS. 3 to 8.

To access compounds represented in FIG. 1A, a synthetic pathway can be followed, as illustrated in FIG. 4. Commercially available 1-(1,3-benzodioxol-5-yl) propan-1-one (5) is subjected to an α-bromination, by dissolving 5 in a suitable solvent such as DCM and treated with bromine (as illustrated in FIG. 4). When the reaction is complete the mixture is washed with water and the organic layer is dried and evaporated to dryness. The residual product 6 can either be recrystallized or used directly for the next step when pure enough. Next, the obtained α-bromo ketone 6 is dissolved in an inert solvent such as tetrahydrofuran or diethyl ether and a solution of the amine in a suitable solvent like ethanol is added at a suitable temperature of −30 to 50° C., more preferably at 0° C. After a reaction time enough to complete reaction, some of the volatiles are stripped off and the residue is worked up by standard extraction procedures and thereby preventing formation of pyrazine condensation products caused by dimerization. The β-keto amine 7 is converted to its hydrochloride before it is subjected to a direct Wittig reaction with a mixture of methyl-triphenylphosphonium bromide and a base like butyl lithium (BuLi) in a suitable solvent like THF at a temperature of −100° C. to 60° C., more preferably at 0° C. After a reaction time enough to complete reaction, some of the volatiles are stripped off and the residue is worked up by standard extraction procedures. The amine base is then dissolved or kept in a solvent, favorably non-protic, most favorably in TBME, diethyl ether or dioxane, and neutralized by the addition of anhydrous hydrogen chloride or sulfuric acid or any other salt forming organic agent such as fumaric acid, tartaric acid, or acetic acid in a similar solvent. The precipitated amine salt is isolated to get the final amine 8, as outlined in FIG. 4.

To access compounds represented in FIG. 1B, a synthetic pathway can be followed, as illustrated in FIG. 6. The amino acid 19, to be used racemic or enantiomerically pure, is N-BOC protected by allowing it to react with BOC2O in a suitable solvent or in a solvent mixture like water and dioxane and application of a base like NaOH. The product 20 is isolated by standard extraction procedures before it is subjected to an amide coupling reaction in a solvent like DCM with N,O-dimethylhydroxylamine hydrochloride and with a coupling reagent like EDC in the presence of a non-reacting base like N-methylmorpholine (NMM) to form the Weinreb amide 21, which is isolated by standard extraction procedures. Selective reduction of the Weinreb amide to the aldehyde 22 is performed with a reducing agent like LiAlH₄ in an inert solvent like THF at a temperature of −80 to 60° C., more favorably at −15° C. After quenching the reaction mixture with a protic solvent or protic salt solution like aqueous KHSO₄, the aldehyde 22 is isolated by standard extraction procedures. The aldehyde 22 is subjected to a Wittig reaction with a mixture of methyl-triphenylphosphonium bromide and a base like LHMDS in a suitable solvent like THF at a temperature of −100° C. to 60° C., more preferably at −80° C., and then allowed to warm to ambient temperature. After a reaction time enough to complete reaction, the mixture is worked up by standard extraction procedures and purified by a procedure like silica gel chromatography to get the N-protected allylamine 23. Treating 23 with a reagent able to N-deprotect, like HCl in dioxane, leads to the final compound 24 as a salt as outlined in FIG. 6. When reducing the N-BOC group of 23 with a reducing agent like LiAlH₄ in a solvent like THF at temperatures of −10° C. to 70° C. the final N-methylated product 25 can be obtained as shown in FIG. 6.

To access compounds represented in FIG. 1B, a synthetic pathway can also be followed, as illustrated in FIG. 7 and FIG. 8, which uses different starting materials leading to additional examples, namely compounds 35 and 43.

To access compounds represented in FIG. 1C, a synthetic pathway can be followed, as illustrated in FIG. 6. Aldehyde 22, obtained as described above and as outlined in FIG. 6, can be treated with a reagent like dimethyl-(1-diazo-2-oxopropyl) phosphonate in the presence of a base like K₂CO₃ in a solvent like methanol at a temperature of −20° C. to 50° C., more preferably at ambient temperature, and the formed product 26 can be isolated by standard extraction procedures and chromatography. Treating 26 with a reagent able to N-deprotect, like HCl in dioxane, leads to the final compound 27 as a salt as outlined in FIG. 6. Intermediate 26 can also be N-alkylated by treating it with a deprotonating base like NaH and with an alkylating agent like methyl iodide in a solvent like THF, at a temperature of −50° C. to 60° C., more preferably at 0° C. The intermediate obtained and isolated by standard extraction procedures can then be N-deprotected with an agent like HCl in dioxane to get the final N-alkylated product 28, as outlined in FIG. 6. When reducing the N-BOC group of 26 with a reducing agent like LiAlH₄ in a solvent like THF, at temperatures necessary to allow reaction, parts of the triple bond are reduced to the corresponding alkene (unpublished findings).

To access compounds represented in FIG. 1C, a synthetic pathway can also be followed, as illustrated in FIG. 8, which uses a different starting material leading to an additional example, namely compound 37 (DDH-4C-B).

### Example 3—Detailed Description of the Chemical Preparation of the Compounds

General. NMR was performed on a Bruker NMR (¹H: 300 MHz and ¹⁹F: 282 MHz) at ambient temperature. Reaction controls were performed by silica gel TLC (F₂₅₄; UV detection) and HPLC UV DAD & MS (Agilent 1100, Waters SQD). Where not specifically indicated, racemates were used as starting materials.

Preparation of the allylamine-type phenethylamine derivative 8 1-(1,3-Benzodioxol-5-yl)-2-bromopropan-1-one, 6. Bromine (5.10 mL; 100 mmol) was added dropwise under occasional cooling to a solution of 17.82g (100 mol) 1-(1,3-benzodioxol-5-yl) propan-1-one (5) in 160 mL dichloromethane (DCM), whereby large amounts of HBr gas evolved, which were trapped in a gas washing bottle containing ice-cooled aqueous NaOH. After 1 hour the reaction mixture was poured into 400 mL ice-water, and the org. layer was washed with water (4×100 mL), dried over Na₂SO₄, and concentrated in vacuo. Yield: 23.98g (93.3%) 6 as a brown solid. ¹H-NMR (CDCl₃): 1.91 (d, CH₃), 5.22 (q, BrCH), 6.09 (s, OCH₂O), 6.90 (d, 1 arom. H), 7.51 (d, 1 arom. H), 7.67 (dxd, 1 arom. H).

1-(1,3-Benzodioxol-5-yl)-2-(methylamino) propan-1-one hydrochloride, 7. A solution of 5.14g (20 mmol) in 40 mL THF anhydr. was added dropwise to an ice-cooled solution of MeNH2 33% in abs. EtOH (7.5 mL; 60 mmol) over the course of 17 min under nitrogen. After stirring for 5 hours under ice-cooling and 30 minutes at ambient temperature the mixture was very quickly concentrated at no heating in vacuo to approx. ¼ of the initial volume. The residue was diluted with 100 mL water, acidified (2M HCl, pH to approx. 2-3) and washed with tert-butyl methyl ether (MTBE; 3×50 mL). The aq. layer was made basic to pH 11-12 (NaOH 2M) and extracted with MTBE (3×50 mL), and the combined org. extracts were dried over Na₂SO₄. To this was added 1.0 mL isopropanol anhydr. before it was carefully neutralized with anhydr. HCl 2M in Et₂Ounder ice-cooling. The solids formed were filtered off, rinsed with MTBE and dried. Yield: 3.64 g (74.7%) 7 as a white solid. ¹H-NMR (D₂O): 1.56 (d, MeCH), 2.74 (s, NMe), 4.96 (q, CHN), 6.09 (d, OCH2O), 7.00 (d, 1 arom. H), 7.43 (d, 1 arom. H), 7.63 (dxd, 1 arom. H).

2-(1,3-Benzodioxol-5-yl)-3-(methylamino) butene hydrochloride (BME-MDMA), 8. A solution of BuLi (1.6M in hexane, 8.31 mL; 3.0 eq) was added at 5-10° C. within 3 minutes to a suspension of 4.75g (13.29 mmol; 3.0eq) methyltriphenylphosphonium bromide in 150 mL THF anhydr. under nitrogen. After stirring for 75 minutes at this temperature, 1.08 g (4.43 mmol) finely powdered ketone 7 was added all at once by quickly opening and closing the reaction flask. The cooling bath was removed, and the mixture was treated with ultrasonic for 1 minute to ease partial dissolution and then allowed to stir at ambient temperature for 19 hours. The reaction mixture was quenched by the addition of 20 mL water, and the org. volatiles were removed in vacuo. The residue was diluted with 100 ml water, and the biphasic mixture was acidified to pH 2-3 with 2M HCl, washed with MTBE (4×50 mL), made basic (NaOH 2M, to pH 11-12), and extracted with MTBE (3×50mL). The org. extracts were combined and dried over Na₂SO₄, whereby 2 g activated charcoal pellets were added prior filtering off. To the filtrate was added 0.5 mL isopropanol anhydr. before it was carefully neutralized with anhydr. HCl 2M in Et₂O under ice-cooling. The solids formed were filtered off, rinsed with ethyl acetate (EtOAc)/isopropanol 4:1 (10 mL), EtOAc (8 mL) and hexane (10 mL) and finally dried. Yield: 0.742g (69.3%) product 8 as a white solid. 1H-NMR (D2O): 1.49 (d, MeCH), 2.64 (s, NMe), 4.31 (q, CHN), 5.38 (s, 1H, H₂C=C), 5.56 (s, 1H, H₂C=C), 5.97 (s, OCH₂O), 6.86-6.99 (superimposed signals, 3 arom. H).

Preparation of the allylamine-type phenethylamine and tryptamine derivatives 24, 25, 35 and 43.

3-(1,3-Benzodioxol-5-yl)-2-(tert-butoxycarbonylamino) propanoic acid, 20. To a vigorously stirred suspension of 2.29 g (10.95 mmol) 2-amino-3-(1,3-benzodioxol-5-yl) propanoic acid (19) in 110 mL dioxane and 55 mL deion. water was subsequently added 8.2 mL NaOH 2M and 2.44 g (1.02 eq) BOC₂O under ice-cooling. The ice-bath was removed and after stirring for 90 minutes (note: when the reaction is not complete some additional BOC₂O can be used) most of the dioxane was removed in vacuo. The residual liquid was acidified with HCl 1M to pH 3 and the heterogenous mixture was extracted with MTBE (3×50mL). The combined org. extracts were washed with water (2×50mL), dried over Na₂SO₄ and concentrated in vacuo. Yield: 2.74 g (80.9%) 20 as a brownish-white solid. ¹H-NMR (CDCl₃. A rather complex spectrum was obtained): 1.45 (s, t-Bu), 2.80-3.20 (m, ArCH₂), 4.40; 4.59; 4.97; 5.74 (m; sum=2H, NH and CHN), 5.96 (m, OCH₂O), 6.65 (dxd, 1 arom. H), 6.70 (d, 1 arom. H), 6.77 (d, 1 arom. H).

N-(tert-Butoxycarbonyl)-2-amino-3-(1,3-benzodioxol-5-yl)-N-methoxy-N-methylpropionamide, 21. To as suspension of 2.70 g (8.73 mmol) 20 and 0.954 g (9.78 mmol) N,O-dimethylhydroxylamine hydrochloride in 50 mL DCM were subsequently added 1.08 mL (9.78 mmol) N-methylmorpholine (NMM) and, in three portions spaced by 1 minute, 1.80 g (9.38 mmol) N-ethyl-N′-(3-dimethylaminopropyl)-carbodiimide hydrochloride (EDC). After 2 hours the mixture was diluted with 50 mL DCM, successively washed with sat. NH₄Cl solution, sat. NHCO₃ solution and brine (50 mL, each), dried over Na₂SO₄ and concentrated in vacuo. Yield: 3.3 g (quantitative) 21 as an ochre-beige solid. ¹H-NMR (CDCl₃): 1.42 (s, t-Bu), 2.82 (m, 1H, ArCH₂), 2.99 (m, 1H, ArCH₂), 3.20 (s, NMe), 3.73 (s, MeO), 4.89 and 5.17 (m, each; 2H, NH and CHN), 5.94 (m, OCH₂O), 6.63 (dxd, 1 arom. H), 6.68 (d, 1 arom. H), 6.74 (d, 1 arom. H).

tert-Butyl (1-oxo-3-(1,3-benzodioxol-5-yl) propan-2-yl) carbamate, 22. Crude Weinreb amide 21 (3.3 g, contain 8.62 mmol) was dissolved in 50 mL THF anhydr. under nitrogen by applying ultrasonic and gentle warming before the solution was cooled with an ice-NaCl cooling bath to about −15° C. Next, 4.31 mL (4.31 mmol) LiAlH₄ 1M in THF were added dropwise (12 minutes). After completion of addition the mixture was briefly allowed to reach 0° C. (for about 5 minutes) and then cooled again to −15° C. A 25% KHSO₄ solution (100 mL) was added over 10 seconds and the ice-bath was removed. The mixture was stirred for 15 minutes to reach ambient temperature. Next, 100 ml EtOAc were added, and after stirring vigorously for 5 min the layers were separated, the org. layer washed with sat. NaHCO₃ solution (1×50mL) and brine (1×50mL), dried over Na₂SO₄ and concentrated in vacuo. Yield: 2.40 g (94.9%) 22 as an orange sticky mass. To be stored in the fridge. ¹H-NMR (CDCl₃): 1.47 (s, t-Bu), 3.06 (d, ArCH₂), 4.39 and 5.06 (m, each; 2H, NH and CHN), 5.96 (m, OCH₂O), 6.63 (dxd, 1 arom. H), 6.68 (d, 1 arom. H), 6.77 (d, 1 arom. H), 9.64 (s, CHO).

tert-Butyl (1-(1,3-benzodioxol-5-yl) but-3-en-2-yl) carbamate, 23. A solution of lithium bis(trimethylsilyl)amide (LHMDS) in THF (1M, 17.42 mL; 2.2 eq) was added (3 min) to an ice-cooled suspension of 6.52 g (18.19 mmol; 2.3 eq) methyl triphenylphosphonium bromide in 80 mL THF anh. under nitrogen. After stirring for 2.5 hours the mixture was cooled to −78° C. and a solution of 2.32 g (7.91 mmol) aldehyde 22 in 32 mL THF anhydr. was added over the course of 12 minutes. The cooling bath was removed and stirring at ambient temperature was maintained for 16 hours. Next, 60 mL hexane were added and the suspension was directly filtered off, the filtrate diluted with 4 mL methanol and concentrated in vacuo. The residue was partitioned between 80 mL EtOAc and 50 mL water, the organic layer was further washed with 40 mL water, dried over Na₂SO₄ and concentrated in vacuo to get 4.62 g crude 23 as a viscous oil. The oil was purified by silica gel column (hexane/EtOAc=95/5); elution with hexane/EtOAc=95/5 and, towards the end, with 9/1. Yield: 674 mg (29.3%) product 23 as a white solid. ¹H-NMR (CDCl₃): 1.44 (s, t-Bu), 2.78 (d, ArCH₂), 4.43 (m, 2H, superimposition of NH and CHN), 5.10 (m, 1H, HC=CH₂), 5.15 (dxm, 1H, HC=CH₂), 5.81 (m, 1H, HC=CH₂), 5.95 (m, OCH₂O), 6.65 (dxd, 1 arom. H), 6.70 (d, 1 arom. H), 6.76 (d, 1 arom. H).

1-(1,3-Benzodioxol-5-yl) but-3-en-2-amine hydrochloride (DH-BDB), 24. To a solution of 85 mg (0.292 mmol) 23 in 0.5 mL dioxane anhydr. was added 1.5 mL anhydr. HCl 4M in dioxane under nitrogen. After stirring for 75 minutes the volatiles were removed in vacuo (purging the rotary evaporator with nitrogen) and the residual solid was rinsed with diethyl ether anhydr. (3×1mL) and finally dried in vacuo. Yield: 58.2 mg (87.5%) 24 as a white solid. ¹H-NMR (D₂O): 2.94 (m, ArCH₂), 3.99 (m, CHN), 5.30 (m, 2H, HC=CH₂), 5.85 (m, 1H, HC=CH₂), 5.93 (s, OCH₂O), 6.75 (dxd, 1 arom. H), 6.81 (d, 1 arom. H), 6.85 (d, 1 arom. H).

N-Methyl-1-(1,3-benzodioxol-5-yl) but-3-en-2-amine hydrochloride (DH-MBDB), 25. A solution of 580 mg (1.99 mmol) 23 in 10 mL THF anhydr. was added within 2 minutes to 4.38 mL (2.2 eq) LiAlH₄ 1M in THF anhydr. under nitrogen. Next the mixture was heated to a gentle reflux for 3 hours. The formed suspension was cooled with an ice bath and quenched cautiously with dropwise addition 0.69 mL isopropanol and 0.53 mL NaOH 2M aq. The suspension was filtered off, rinsed with additional THF and the combined filtrates were concentrated in vacuo. The residual colorless oil (385 mg) was dissolved in 30mL diethyl ether anhydr. containing 0.1 mL isopropanol and carefully neutralized by the addition of HCl 2 M anhydr. in diethyl ether. The white suspension was filtered off, rinsed with diethyl ether, and dried. Yield: 331 mg (68.8%) 25 as a white solid. ¹H-NMR (D₂O): 2.61 (s, MeN), 2.98 (m, ArCH₂), 3.83 (m, CHN), 5.40 (m, 2H, HC=CH₂), 5.71 (m, 1H, HC=CH₂), 5.92 (s, OCH₂O), 6.74 (dxd, 1 arom. H), 6.79 (d, 1 arom. H), 6.84 (d, 1 arom. H).

2-Amino-3-(4-bromo-2,5-dimethoxyphenyl) propanoic acid, 30. A suspension of 2.37 g (9.06 mmol) 2-amino-3-(2,5-dimethoxyphenyl) propanoic acid hydrochloride (29; commercially available. ¹H-NMR (DMSO-D₆): 3.06 (m, ArCH₂), 3.70 (s, MeO), 3.74 (s, MeO), 4.01 (t, CHN), 6.86 (m, 3 arom. H)) in 75 mL glacial acetic acid was heated to 95° C. until all went into solution. Next, a solution of 0.486 mL (9.51 mmol) bromine in 9 mL acetic acid was added within 5 minutes and heating was continued for 1 hour. The mixture was cooled to about 10° C. and the suspension was filtered off, rinsed with acetic acid (2×10 mL) and MTBE (3×15 mL) and the solids were dried. Yield: 2.54 g (83.1%, based on HCl salt assumption) 30 as a beige solid. ¹H-NMR (DMSO-D₆): 3.06 (m, ArCH₂), 3.77 (s, MeO), 3.79 (s, MeO), 4.08 (t, CHN), 7.03 (s, 1 arom. H), 7.21 (s, 1 arom. H), 8.21 (bs, NH3⁺), 13.76 (bs, COOH).

3-(4-Bromo-2,5-dimethoxyphenyl)-2-(tert-butoxycarbonylamino) propanoic acid, 31. It was followed the procedure described for the preparation of compound 20. From 2.54 g (7.46 mmol; salt assumed: HCl) 30, 1.66 g (7.83 mmol) BOC₂O and 5.6 mL NaOH 2M in 75 mL dioxane and 35 mL water were obtained 2.53 g (83.9%) 31 as a white solid. ¹H-NMR (CDCl₃): 1.42 (s, t-Bu), 3.11 (m, ArCH₂), 3.81 (s, MeO), 3.86 (s, MeO), 4.50 and 5.31 (m, each; 2H, NH and CHN), 6.77 (s, 1 arom. H), 7.08 (s, 1 arom. H). N-(tert-Butoxycarbonyl)-2-amino-3-(4-bromo-2,5-dimethoxyphenyl)-N-methoxy-N-methylpropionamide, 32. It was followed the procedure described for the preparation of compound 21. From 2.50 g (6.18 mmol) 31, 0.675 g (6.92 mmol) N,O-dimethylhydroxylamine hydrochloride, 0.765 mL (6.92 mmol) NMM and 1.27 g (6.64 mmol) EDC in 35 mL DCM were obtained 2.72 g (98.6%) 32 as a beige solid. ¹H-NMR (CDCl₃): 1.39 (s, t-Bu), 2.97 (m, ArCH₂), 3.20 (s, NMe), 3.77 (m, MeON), 3.82 (s, MeO), 3.87 (s, MeO), 4.99 and 5.26 (m, each; 2H, NH and CHN), 6.72 (s, 1 arom. H), 7.04 (s, 1 arom. H).

tert-Butyl (1-oxo-3-(4-bromo-2,5-dimethoxyphenyl) propan-2-yl)carbamate, 33. It was followed the procedure described for the preparation of compound 22. From 1.35 g (3.02 mmol) 32 in 30 mL THF and 1.51 mL (1.51 mmol) 1M LiAlH₄ in THF were obtained 1.15 g (98.3%) 33 as a beige solid. ¹H-NMR (CDCl₃): 1.44 (s, t-Bu), 3.13 (d, ArCH₂), 3.78 (s, MeO), 3.86 (s, MeO), 4.42 and 5.23 (m, each; 2H, NH and CHN), 6.73 (s, 1 arom. H), 7.06 (s, 1 arom. H), 9.59 (s, CHO).

tert-Butyl (1-(4-bromo-2,5-dimethoxyphenyl)but-3-en-2-yl) carbamate, 34. It was followed the procedure described for the preparation of compound 23. From 1.14 g (2.94 mmol) 33 in 12 mL THF, 2.42 g (2.3 eq) methyl triphenylphosphonium bromide in 30 mL THF and 6.47 mL (2.2 eq) LHMDS 1M in THF were obtained 426 mg (37.5%) 34 as a white solid. ¹H-NMR (CDCl₃): 1.39 (s, t-Bu), 2.83 (m, ArCH₂), 3.82 (s, MeO), 3.87 (s, MeO), 4.39 and 4.73 (bs, each; 2H, NH and CHN), 5.14 (m, 2H, HC=CH₂), 5.84 (m, 1H, HC=CH₂), 6.75 (s, 1 arom. H), 7.06 (s, 1 arom. H).

1-(4-Bromo-2,5-dimethoxyphenyl)but-3-en-2-amine hydrochloride (DH-4C-B), 35. It was followed the procedure described for the preparation of compound 24. From 420 mg (1.09 mmol) 34 in 2 mL dioxane anhydr. and 5.6 mL HCl 4M in dioxane anhydr. were obtained 313 mg (88.9%) 35 as a white solid. ¹H-NMR (D₂O): 2.97 (m, ArCH₂), 3.77 (s, MeO), 3.80 (s, MeO), 4.05 (q, CHN), 5.27 (m, 2H, HC=CH₂), 5.83 (m, 1H, HC=CH₂), 6.91 (s, 1 arom. H), 7.24 (s, 1 arom. H).

N-(tert-Butoxycarbonyl)-2-amino-3-(indol-3-yl)-N-methoxy-N-methylpropionamide, 40. It was followed the procedure described for the preparation of compound 21. From 2.66 g (8.73 mmol) N-(tert-butoxycarbonyl)-D-L-tryptophan (39; this is commercially available but can also easily be prepared from D-L-tryptophan (38) by following the procedure described for compound 20), 0.954 g (9.78 mmol) N,O-dimethylhydroxylamine hydrochloride, 1.08 mL (9.78 mmol) NMM and 1.80 g (9.38 mmol) EDC in 50 mL DCM were obtained 3.14 g (quantitative) 40 as a white solid. ¹H-NMR (CDCl₃): 1.05-1.50 (m, t-Bu), 3.17 (s, superimposed, NMe), 3.20 (m, superimposed, 2H, ArCH₂), 3.67 (s, MeO), 5.02 and 5.25 (m, each; 2H, NH and CHN), 7.07 (d, 1 arom. H), 7.17 (m, 2 arom. H), 7.37 (d, 1 arom. H), 7.63 (d, 1 arom. H), 8.08 (bs, indole-NH).

tert-Butyl (1-oxo-3-(indol-3-yl)propan-2-yl)carbamate, 41. It was followed the procedure described for the preparation of compound 22. From Weinreb amide 40 (1.55 g, contain 4.37 mmol) in 45 mL THF and 2.23 mL (2.23 mmol) 1M LiAlH4 in THF were obtained 1.26 g (100%) 41 as a yellow mass that slowly crystallized to a beige solid. ¹H-NMR (CDCl₃): 1.47 (s, t-Bu), 3.29 (m, ArCH₂), 4.54 and 5.18 (m, each; 2H, NH and CHN), 7.07 (d, 1 arom. H), 7.12-7.27 (m, 2 arom. H), 7.40 (d, 1 arom. H), 7.63 (d, 1 arom. H), 8.15 (bs, indole-NH), 9.67 (s, CHO).

tert-Butyl (1-(indol-3-yl)but-3-en-2-yl)carbamate, 42. It was followed the procedure described for the preparation of compound 23. From 1.25 g (4.33 mmol) 41 in 18 mL THF, 3.56 g (2.3 eq) methyl triphenylphosphonium bromide in 43 mL THF and 9.53mL (2.2eq) LHMDS 1M in THF, and after chromatography with a more polar solvent system (hexane/EtOAc 95/5, 90/10 and finally 80/20) were obtained 144 mg (11.6%) 42 as a colorless viscous oil. ¹H-NMR (CDCl₃): 1.43 (s, t-Bu), 3.04 (m, ArCH₂), 4.56 (bs, 2H, superimposed NH and CHN), 5.13 (m, 2H, HC=CH₂), 5.89 (m, 1H, HC=CH₂), 7.07 (d, 1 arom. H), 7.18 (m, 2 arom. H), 7.39 (d, 1 arom. H), 7.65 (d, 1 arom. H), 8.06 (bs, indole-NH).

1-(Indol-3-yl) but-3-en-2-amine hemifumarate (DH-α-ET), 43. It was first followed the procedure described for the preparation of compound 24; the reaction mixture from 140 mg (0.489 mmol) 42 in 1 mL dioxane anhydr. and 2.5 mL HCl 4M in dioxane anhydr., after completion of reaction (LCMS), was diluted with 10 mL ethyl acetate and 5 mL saturated NaHCO₃ solution. After shaking until all solids were dissolved the layers were separated, and the aq. layer further extracted with 10 mL ethyl acetate. The combined org. layers were dried (Na₂SO₄) and concentrated in vacuo. The residue was dissolved in 5 mL diethyl ether anhydr. containing 0.1 mL isopropanol and carefully neutralized to pH 7-8 under nitrogen with a solution of 28.4 mg fumaric acid (theoretical amount to form a hemifumarate) in 7 mL diethyl ether anhydr. containing 0.2 mL isopropanol (ultrasonic helped to dissolve). The formed suspension was filtered off, rinsed with diethyl ether, and dried. Yield: 78.8 mg (66.0%) 43 as a beige solid. ¹H-NMR (D₂O): 3.18 (m, ArCH₂), 4.12 (q, CHN), 5.32 (m, 2H, HC=CH₂), 5.92 (m, 1H, HC=CH₂), 6.46 (s, “1H” (2× 0.5H), HC=CH, from tartaric acid), 7.15 (t, 1 arom. H), 7.24 (t, superimposed, 1 arom. H), 7.27 (s, superimposed, 1 arom. H), 7.49 (d, 1 arom. H), 7.65 (d, 1 arom. H). Preparation of the propargylamine-type derivatives 27, 28 and 37.

tert-Butyl (1-(1,3-benzodioxol-5-yl)but-3-yn-2-yl)carbamate, 26. To a solution of 2.30 g (7.84 mmol) 22 in 118 mL MeOH anhydr. was added subsequently 2.17 g (15.68 mmol; 2.0 eq) K₂CO₃ and 1.81 g (9.41 mmol; 1.2 eq) dimethyl 1-diazo-2-oxopropylphosphonate under nitrogen. After stirring for 2.5 hours about ½ of the MeOH volume was stripped off and the residual mixture was diluted with MTBE, washed with saturated NaHCO3 solution (1×100 mL), water (2×80 mL) and brine (1×50 mL) and finally dried (Na₂SO₄) and concentrated in vacuo. The residue (2.34 g) was purified by using a small silica gel chromatography (hexane/EtOAc=93/7). Yield: 1.90 g (83.7%) 26 as a white solid. ¹H-NMR (CDCl₃): 1.46 (s, t-Bu), 2.31 (d, C=CH), 2.93 (m, ArCH₂), 4.64 and 4.68 (two bs, 2H, superimposed NH and CHN), 5.96 (m, OCH₂O), 6.73 (dxd, 1 arom. H), 6.78 (d, 1 arom. H), 6.81 (d, 1 arom. H).

1-(1,3-Benzodioxol-5-yl)but-3-yn-2-amine hydrochloride (DDH-BDB), 27. To a solution of 700 mg (2.42 mmol) 26 in 6 mL dioxane anhydr. were added 12 mL anhydr. HCl 4M in dioxane under nitrogen. After stirring for 6 hours 20 mL diethyl ether anhydr. were added and the suspension was filtered off, and the filter cake was rinsed with diethyl ether anhydr. (3×5mL) and finally dried in vacuo. Yield: 486 mg (89.0%) 27 as a white solid. ¹H-NMR (D₂O): 2.97 (d, C=CH), 3.07 (m, ArCH₂), 4.31 (m, CHN), 5.94 (s, OCH₂O), 6.82 (dxd, 1 arom. H), 6.87 (d, 1 arom. H), 6.89 (d, 1 arom. H).

N-Methyl-1-(1,3-benzodioxol-5-yl)but-3-yn-2-amine hydrochloride (DDH-MBDB), 28. To an ice-cooled solution of 1.18 g (4.08 mmol) 26 in 20 mL THF anhydr. was added 196 mg (4.90 mmol) NaH in three portions under nitrogen. After stirring for 10 min 0.305 mL (4.90 mmol) methyl iodide was added and stirring under cooling was continued for 2 hours. Another 0.03 mL methyl iodide was added, and after stirring for another 70 minutes 5 g ice were added to the mixture, followed by 20 ml water. Most of the THF was removed in vacuo and the residue was extracted with MTBE (2×40 mL). The extracts were dried (Na₂SO₄) and concentrated in vacuo. Yield: 1.15 g (92.7%) of the N-methylated N-BOC intermediate. ¹H-NMR (CDCl₃. A complex spectrum was observed): 1.39 (bm, t-Bu), 2.36 (bs, C=CH), 2.89 (m, ArCH₂), 5.02 and 5.21 (two bs, 2H, NH and CHN), 5.94 (m, OCH₂O), 6.60-6.82 (bm, 3 arom. H). The N-methylated N-BOC intermediate was dissolved in 2 mL dioxane anhydr. and 18 mL anhydr. HCl 4M in dioxane was added under nitrogen. After stirring for 1 hour the mixture was concentrated in vacuo. The residual sticky oil was triturated in 20 mL diethyl ether anhydr. to get a fine white suspension, which was filtered off, and the filter cake was rinsed with diethyl ether anhydr. (2×10mL) and finally dried in vacuo. Yield: 793 mg (88.8%) 28 as a white solid. ¹H-NMR (D₂O): 2.79 (s, NMe), 3.07 (d, superimposed, C=CH), 3.10 (m, ArCH₂), 4.29 (m, CHN), 5.94 (s, OCH₂O), 6.82 (dxd, 1 arom. H), 6.86 (d, 1 arom. H), 6.89 (d, 1 arom. H).

tert-Butyl (1-(4-bromo-2,5-dimethoxyphenyl)but-3-yn-2-yl)carbamate, 36. It was followed the procedure described for the preparation of compound 26. From 1.05 g (2.70 mmol) 33, 0.749 g (5.40 mmol; 2.0 eq) K₂CO₃ and 0.624 g (3.24 mmol; 1.2 eq) dimethyl 1-diazo-2-oxopropylphosphonate in 41 mL MeOH anhydr. and final chromatography (hexane/EtOAc=4/1) were obtained 0.921 g (88.8%) 36 as a white solid. ¹H-NMR (CDCl₃): 1.41 (s, t-Bu), 2.29 (d, C=CH), 2.99 (m, ArCH₂), 3.82 (s, MeO), 3.87 (s, MeO), 4.71 and 4.87 (two bs, 2H, NH and CHN), 6.82 (s, 1 arom. H), 7.07 (s, 1 arom. H).

1-(4-Bromo-2,5-dimethoxyphenyl)but-3-yn-2-amine hydrochloride (DDH-4C-B), 37. The N-BOC compound 36 (0.910 g; 2.37 mmol) was dissolved in 3 mL dioxane anhydr. and 12 mL anhydr. HCl 4M in dioxane was added under nitrogen. After stirring for 1 hour the mixture was concentrated in vacuo until crystallization began. The residue was triturated in 10 mL diethyl ether anhydr. to get a fine white suspension, which was filtered off, and the filter cake was rinsed with diethyl ether anhydr. (3×5 mL) and finally dried in vacuo. Yield: 638 mg (84.0%) 37 as a white solid. ¹H-NMR (D₂O): 2.93 (d, C=CH), 3.08 (m, ArCH₂), 3.74 (s, MeO), 3.79 (s, MeO), 4.38 (dxt, CHN), 6.99 (s, 1 arom. H), 7.23 (s, 1 arom. H).

Throughout this application, various publications, including United States patents, are referenced by author and year and patents by number. Full citations for the publications are listed herein. The disclosures of these publications and patents in their entireties are hereby incorporated by reference into this application in order to more fully describe the state of the art to which this invention pertains.

The invention has been described in an illustrative manner and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Arai, Y., Toyoshima, Y., & Kinemuchi, H. (1986). Studies of monoamine oxidase and semicarbazide-sensitive amine oxidase. II. Inhibition by alpha-methylated substrate-analogue monoamines, alpha-methyltryptamine, alpha-methylbenzylamine and two enantiomers of alpha-methylbenzylamine. Jpn J Pharmacol, 41(2), 191-197. doi: 10.1254/jp.41.191
2. Becker, A. M., Klaiber, A., Holze, F., Istampoulouoglou, I., Duthaler, U., Varghese, N., . . . Liechti, M. E. (2022). Ketanserin reverses the acute response to LSD in a randomized, double-blind, placebo-controlled, crossover study in healthy subjects. Int J Neuropsychopharmacoldoi:10.1093/ijnp/pyac075
3. Blacker, J., Roy, M., Hariharan, S., Headley, C., Upare, A., Jagrap, A., . . . Kadam, N. (2011). Convenient Method for Synthesis of N-Protected α-Amino Epoxides: Key Intermediates for HIV Protease Inhibitors. Org Process Res Dev, 15(2), 331-338.
4. Blough, B. E., Landavazo, A., Partilla, J. S., Decker, A. M., Page, K. M., Baumann, M. H., & Rothman, R. B. (2014). Alpha-ethyltryptamines as dual dopamine-serotonin releasers. Bioorg Med Chem Lett, 24(19), 4754-4758. doi:10.1016/j.bmcl.2014.07.062
5. Bogenschutz, M. P. (2013). Studying the effects of classic hallucinogens in the treatment of alcoholism: rationale, methodology, and current research with psilocybin. Curr Drug Abuse Rev, 6(1), 17-29. Retrieved from http://www.ncbi.nlm.nih.gov/pubmed/23627783
6. Bogenschutz, M. P., Forcehimes, A. A., Pommy, J. A., Wilcox, C. E., Barbosa, P. C., & Strassman, R. J. (2015). Psilocybin-assisted treatment for alcohol dependence: a proof-of-concept study. J Psychopharmacol, 29(3), 289-299. doi: 10.1177/0269881114565144
7. Bogenschutz, M. P., Ross, S., Bhatt, S., Baron, T., Forcehimes, A. A., Laska, E., . . . Worth, L. (2022). Percentage of heavy drinking days following psilocybin-assisted psychotherapy vs placebo in the treatment of adult patients with alcohol use disorder: a randomized clinical trial. JAMA Psychiatry, 79(10), 953-962. doi:10.1001/jamapsychiatry.2022.2096
8. Braden, M. R., & Nichols, D. E. (2007). Assessment of the roles of serines 5.43(239) and 5.46(242) for binding and potency of agonist ligands at the human serotonin 5-HT2A receptor. Mol Pharmacol, 72(5), 1200-1209. doi: 10.1124/mol.107.039255
9. Carhart-Harris, R., Giribaldi, B., Watts, R., Baker-Jones, M., Murphy-Beiner, A., Murphy, R., . . . Nutt, D. J. (2021). Trial of psilocybin versus escitalopram for depression. N Engl J Med, 384(15), 1402-1411. doi:10.1056/NEJMoa2032994
10. Carhart-Harris, R. L., Bolstridge, M., Rucker, J., Day, C. M., Erritzoe, D., Kaelen, M., . . . Nutt, D. J. (2016). Psilocybin with psychological support for treatment-resistant depression: an open-label feasibility study. Lancet Psychiatry, 3, 619-627. doi:10.1016/S2215-0366(16)30065-7
11. Carhart-Harris, R. L., Kaelen, M., Bolstridge, M., Williams, T. M., Williams, L. T., Underwood, R., . . . Nutt, D. J. (2016). The paradoxical psychological effects of lysergic acid diethylamide (LSD). Psychol Med, 46, 1379-1390. doi:10.1017/S0033291715002901
12. Cunningham, M. J., Bock, H. A., Serrano, I. C., Bechand, B., Vidyadhara, D. J., Bonniwell, E. M., . . . Sames, D. (2023). Pharmacological Mechanism of the Non-hallucinogenic 5-HT (2A) Agonist Ariadne and Analogs. ACS Chem Neurosci, 14(1), 119-135. doi:10.1021/acschemneuro.2c00597
13. D'Souza, D. C., Syed, S. A., Flynn, L. T., Safi-Aghdam, H., Cozzi, N. V., & Ranganathan, M. (2022). Exploratory study of the dose-related safety, tolerability, and efficacy of dimethyltryptamine (DMT) in healthy volunteers and major depressive disorder. Neuropsychopharmacology, 47(10), 1854-1862. doi:10.1038/s41386-022-01344-y
14. Danforth, A. L., Grob, C. S., Struble, C., Feduccia, A. A., Walker, N., Jerome, L., . . . Emerson, A. (2018). Reduction in social anxiety after MDMA-assisted psychotherapy with autistic adults: a randomized, double-blind, placebo-controlled pilot study. Psychopharmacology (Berl), 235(11), 3137-3148. doi:10.1007/s00213-018-5010-9
15. Danforth, A. L., Struble, C. M., Yazar-Klosinski, B., & Grob, C. S. (2016). MDMA-assisted therapy: A new treatment model for social anxiety in autistic adults. Prog Neuropsychopharmacol Biol Psychiatry, 64, 237-249. doi:10.1016/j.pnpbp.2015.03.011
16. Davis, A. K., Barrett, F. S., May, D. G., Cosimano, M. P., Sepeda, N. D., Johnson, M. W., . . . Griffiths, R. R. (2021). Effects of psilocybin-assisted therapy on major depressive disorder: a randomized clinical trial. JAMA Psychiatry, 78(5), 481-489. doi:10.1001/jamapsychiatry.2020.3285
17. Dolder, P. C., Muller, F., Schmid, Y., Borgwardt, S. J., & Liechti, M. E. (2018). Direct comparison of the acute subjective, emotional, autonomic, and endocrine effects of MDMA, methylphenidate, and modafinil in healthy subjects. Psychopharmacology (Berl), 235(2), 467-479. doi:10.1007/s00213-017-4650-5
18. Dolder, P. C., Schmid, Y., Mueller, F., Borgwardt, S., & Liechti, M. E. (2016). LSD acutely impairs fear recognition and enhances emotional empathy and sociality. Neuropsychopharmacology, 41, 2638-2646.
19. Dolder, P. C., Schmid, Y., Steuer, A. E., Kraemer, T., Rentsch, K. M., Hammann, F., & Liechti, M. E. (2017). Pharmacokinetics and pharmacodynamics of lysergic acid diethylamide in healthy subjects. Clin Pharmacokinetics, 56, 1219-1230.
20. Dominguez-Clave, E., Soler, J., Elices, M., Pascual, J. C., Alvarez, E., de la Fuente Revenga, M., . . . Riba, J. (2016). Ayahuasca: Pharmacology, neuroscience and therapeutic potential. Brain Res Bull, 126(Pt 1), 89-101. doi:10.1016/j.brainresbull.2016.03.002
21. Dong, C., Ly, C., Dunlap, L. E., Vargas, M. V., Sun, J., Hwang, I. W., . . . Tian, L. (2021). Psychedelic-inspired drug discovery using an engineered biosensor. Cell, 184, 2779-2792.e2718. doi:10.1016/j.cell.2021.03.043
22. Dos Santos, R. G., Osorio, F. L., Crippa, J. A., Riba, J., Zuardi, A. W., & Hallak, J. E. (2016). Antidepressive, anxiolytic, and antiaddictive effects of ayahuasca, psilocybin and lysergic acid diethylamide (LSD): a systematic review of clinical trials published in the last 25 years. Ther Adv Psychopharmacol, 6(3), 193-213. doi: 10.1177/2045125316638008
23. Dumont, G. J., Sweep, F. C., van der Steen, R., Hermsen, R., Donders, A. R., Touw, D. J., . . . Verkes, R. J. (2009). Increased oxytocin concentrations and prosocial feelings in humans after ecstasy (3,4-methylenedioxymethamphetamine) administration. Soc Neurosci, 4(4), 359-366. Retrieved from http:/www.ncbi.nlm.nih.gov/entrez/query.fcgi?cmd=Retrieve&db=PubMed?dopt=Citatio n&list_uids=19562632
24. Francis, S. M., Kirkpatrick, M. G., de Wit, H., & Jacob, S. (2016). Urinary and plasma oxytocin changes in response to MDMA or intranasal oxytocin administration. Psychoneuroendocrinology, 74, 92-100. doi:10.1016/j.psyneuen.2016.08.011
25. Gasser, P., Holstein, D., Michel, Y., Doblin, R., Yazar-Klosinski, B., Passie, T., & Brenneisen, R. (2014). Safety and efficacy of lysergic acid diethylamide-assisted psychotherapy for anxiety associated with life-threatening diseases. J Nerv Ment Dis, 202(7), 513-520. doi:10.1097/NMD.0000000000000113
26. Gasser, P., Kirchner, K., & Passie, T. (2015). LSD-assisted psychotherapy for anxiety associated with a life-threatening disease: a qualitative study of acute and sustained subjective effects. J Psychopharmacol, 29(1), 57-68. doi:10.1177/0269881114555249
27. Glennon, R. A., Raghupathi, R., Bartyzel, P., Teitler, M., & Leonhardt, S. (1992). Binding of phenylalkylamine derivatives at 5-HT1C and 5-HT2 serotonin receptors: evidence for a lack of selectivity. J Med Chem, 35(4), 734-740. Retrieved from http://www.ncbi.nlm.nih.gov/pubmed/1542100
28. Goodwin, G. M., Aaronson, S. T., Alvarez, O., Arden, P. C., Baker, A., Bennett, J. C., . . . Malievskaia, E. (2022). Single-dose psilocybin for a treatment-resistant episode of major depression. N Engl J Med, 387(18), 1637-1648. doi:10.1056/NEJMoa2206443
29. Griffiths, R., Richards, W., Johnson, M., McCann, U., & Jesse, R. (2008). Mystical-type experiences occasioned by psilocybin mediate the attribution of personal meaning and spiritual significance 14 months later. J Psychopharmacol, 22(6), 621-632. doi:10.1177/0269881108094300
30. Griffiths, R. R., Johnson, M. W., Carducci, M. A., Umbricht, A., Richards, W. A., Richards, B. D., . . . Klinedinst, M. A. (2016). Psilocybin produces substantial and sustained decreases in depression and anxiety in patients with life-threatening cancer: a randomized double-blind trial. J Psychopharmacol, 30(12), 1181-1197. doi: 10.1177/0269881116675513
31. Grob, C. S., Danforth, A. L., Chopra, G. S., Hagerty, M., Mckay, C. R., Halberstadt, A. L., & Greer, G. R. (2011). Pilot study of psilocybin treatment for anxiety in patients with advanced-stage cancer. Arch Psychiatry, Gen 68(1), 71-78. doi:10.1001/archgenpsychiatry.2010.116
32. Holze, F., Avedisian, I., Varghese, N., Eckert, A., & Liechti, M. E. (2021). Role of the 5-HT2A receptor in acute effects of LSD on empathy and circulating oxytocin. Front Pharmacol, 12, 711255. doi:10.3389/fphar.2021.711255
33. Holze, F., Duthaler, U., Vizeli, P., Muller, F., Borgwardt, S., & Liechti, M. E. (2019). Pharmacokinetics and subjective effects of a novel oral LSD formulation in healthy subjects. Br J Clin Pharmacol, 85, 1474-1483. doi:10.1111/bcp.13918
34. Holze, F., Gasser, P., Muller, F., Dolder, P. C., & Liechti, M. E. (2023). Lysergic acid diethylamide-assisted therapy in patients with anxiety with and without a life-threatening illness: a randomized, double-blind, placebo-controlled phase II study. Biol Psychiatry, 93(3), 215-223. doi: 10.1016/j.biopsych.2022.08.025
35. Holze, F., Ley, L., Muller, F., Becker, A. M., Straumann, I., Vizeli, P., . . . Liechti, M. E. (2022). Direct comparison of the acute effects of lysergic acid diethylamide and psilocybin in a double-blind placebo-controlled study in healthy subjects. Neuropsychopharmacology, 47(6), 1180-1187. doi: 10.1038/s41386-022-01297-2
36. Holze, F., Vizeli, P., Ley, L., Muller, F., Dolder, P., Stocker, M., . . . Liechti, M. E. (2021). Acute dose-dependent effects of lysergic acid diethylamide in a double-blind placebo-controlled study in healthy subjects. Neuropsychopharmacology, 46(3), 537-544. doi:10.1038/s41386-020-00883-6
37. Holze, F., Vizeli, P., Muller, F., Ley, L., Duerig, R., Varghese, N., . . . Liechti, M. E. (2020). Distinct acute effects of LSD, MDMA, and D-amphetamine in healthy subjects. Neuropsychopharmacology, 45(3), 462-471. doi:10.1038/s41386-019-0569-3
38. Hysek, C. M., Schmid, Y., Simmler, L. D., Domes, G., Heinrichs, M., Eisenegger, C., . . . Liechti, M. E. (2014). MDMA enhances emotional empathy and prosocial behavior. Soc Cogn Affect Neurosci, 9, 1645-1652. doi:10.1093/scan/nst161
39. Hysek, C. M., Simmler, L. D., Nicola, V., Vischer, N., Donzelli, M., Krähenbühl, S., . . . Liechti, M. E. (2012). Duloxetine inhibits effects of MDMA (“ecstasy”) in vitro and in humans in a randomized placebo-controlled laboratory study. PLOS One, 7, e36476.
40. Ichikawa, J., & Meltzer, H. Y. (2000). The effect of serotonin (1A) receptor agonism on antipsychotic drug-induced dopamine release in rat striatum and nucleus accumbens. Brain Res, 858(2), 252-263. doi:10.1016/s0006-8993(99)02346-x
41. Iversen, L. (2013). Volume 14: Affective Disorders: Drug Actions in Animals and Man: Springer
42. Janowsky, A., Eshleman, A. J., Johnson, R. A., Wolfrum, K. M., Hinrichs, D. J., Yang, J., . . . Riscoe, M. K. (2014). Mefloquine and psychotomimetics share neurotransmitter receptor and transporter interactions in vitro. Psychopharmacology (Berl), 231(14), 2771-2783. doi:10.1007/s00213-014-3446-0
43. Janssen, A. P. A., van Hengst, J. M. A., Bequignon, O. J. M., Deng, H., van Westen, G. J. P., & van der Stelt, M. (2019). Structure Kinetics Relationships and Molecular Dynamics Show Crucial Role for Heterocycle Leaving Group in Irreversible Diacylglycerol Lipase Inhibitors. J Med Chem, 62 (17), 7910-7922. doi:10.1021/acs.jmedchem.9b00686
44 Johnson, M. W., Garcia-Romeu, A., Cosimano, M. P., & Griffiths, R. R. (2014). Pilot study of the 5-HT2AR agonist psilocybin in the treatment of tobacco addiction. J Psychopharmacol, 28(11), 983-992. doi:10.1177/0269881114548296
45. Johnson, M. W., Garcia-Romeu, A., & Griffiths, R. R. (2016). Long-term follow-up of psilocybin-facilitated smoking cessation. Am J Drug Alcohol Abuse, 43, 55-60. doi:10.3109/00952990.2016.1170135
46. Kazuhiko, T., Yuji, H., Koichiro, O., & Hitosi, N. (1980). Wittig-type Reaction of Dimetallated Carbodianion Species as Produced by Zinc Reduction of gem-Polyhalogen Compounds in the Presence of Lewis Acids. Bulletin of the Chemical Society of Japan, 53, 1698-1702.
47. Kirkpatrick, M. G., Lee, R., Wardle, M. C., Jacob, S., & de Wit, H. (2014). Effects of MDMA and intranasal oxytocin on social and emotional processing. Neuropsychopharmacology, 39, 1654-1663. doi:10.1038/npp.2014.12
48. Krebs, T. S., & Johansen, P. O. (2012). Lysergic acid diethylamide (LSD) for alcoholism: meta-analysis of randomized controlled trials. J Psychopharmacol, 26(7), 994-1002. doi:10.1177/0269881112439253
49. Liechti, M. (2015). Novel psychoactive substances (designer drugs): overview and pharmacology of modulators of monoamine signaling. Swiss Med Wkly, 145, w14043. doi:10.4414/smw.2015.14043
50. Liechti, M. E. (2017). Modern clinical research on LSD. Neuropsychopharmacology, 42, 2114-2127.
51. Luethi, D., & Liechti, M. E. (2018). Monoamine transporter and receptor interaction profiles in vitro predict reported human doses of novel psychoactive stimulants and psychedelics. Int J Neuropsychopharmacol, 21(10), 926-931. doi:10.1093/ijnp/pyy047
52. Ly, C., Greb, A. C., Cameron, L. P., Wong, J. M., Barragan, E. V., Wilson, P. C., . . . Olson, D. E. (2018). Psychedelics promote structural and functional neural plasticity. Cell Rep, 23(11), 3170-3182. doi:10.1016/j.celrep.2018.05.022
53. Madsen, M. K., Fisher, P. M., Burmester, D., Dyssegaard, A., Stenbaek, D. S., Kristiansen, S., . . . Knudsen, G. M. (2019). Correction: Psychedelic effects of psilocybin correlate with serotonin 2A receptor occupancy and plasma psilocin levels. Neuropsychopharmacology, 44(7), 1328-1334. doi:10.1038/s41386-019-0360-5
54. Marona-Lewicka, D., Thisted, R. A., & Nichols, D. E. (2005). Distinct temporal phases in the behavioral pharmacology of LSD: dopamine D2 receptor-mediated effects in the rat and implications for psychosis. Psychopharmacology (Berl), 180(3), 427-435. doi:10.1007/s00213-005-2183-9
55. Mitchell, J. M., Bogenschutz, M., Lilienstein, A., Harrison, C., Kleiman, S., Parker-Guilbert, K., . . . Doblin, R. (2021). MDMA-assisted therapy for severe PTSD: a randomized, double-blind, placebo-controlled phase 3 study. Nat Med, 27(6), 1025-1033. doi:10.1038/s41591-021-01336-3
56. Mithoefer, M. C., Feduccia, A. A., Jerome, L., Mithoefer, A., Wagner, M., Walsh, Z., . . . Doblin, R. (2019). MDMA-assisted psychotherapy for treatment of PTSD: study design and rationale for phase 3 trials based on pooled analysis of six phase 2 randomized controlled trials. Psychopharmacology (Berl) doi:10.1007/s00213-019-05249-5
57. Mithoefer, M. C., Wagner, M. T., Mithoefer, A. T., Jerome, I., & Doblin, R. (2010). The safety and efficacy of ±3,4-methylenedioxymethamphetamine-assisted psychotherapy in subjects with chronic, treatment-resistant posttraumatic stress disorder: the first randomized controlled pilot study. J Psychopharmacol, 25, 439-452. doi:10.1177/0269881110378371
58. Nagai, F., Nonaka, R., & Satoh Hisashi Kamimura, K. (2007). The effects of non-medically used psychoactive drugs on monoamine neurotransmission in rat brain. Eur J Pharmacol, 559(2-3), 132-137. doi:10.1016/j.ejphar.2006.11.075
59. Nonaka, R., Nagai, F., Ogata, A., & Satoh, K. (2007). In vitro screening of psychoactive drugs by [(35)S]GTPgammaS binding in rat brain membranes. Biol Pharm Bull, 30(12), 2328-2333. doi:10.1248/bpb.30.2328
60. Oehen, P., Traber, R., Widmer, V., & Schnyder, U. (2013). A randomized, controlled pilot study of MDMA (±3,4-methylenedioxymethamphetamine)-assisted psychotherapy for treatment of resistant, chronic post-traumatic stress disorder (PTSD). J Psychopharmacol, 27(1), 40-52. doi:10.1177/0269881112464827
61. Oeri, H. E. (2020). Beyond ecstasy: Alternative entactogens to 3,4-methylenedioxymethamphetamine with potential applications in psychotherapy. J Psychopharmacol, 269881120920420. doi:10.1177/0269881120920420
62. Palhano-Fontes, F., Barreto, D., Onias, H., Andrade, K. C., Novaes, M. M., Pessoa, J. A., . . . Araujo, D. B. (2019). Rapid antidepressant effects of the psychedelic ayahuasca in treatment-resistant depression: a randomized placebo-controlled trial. Psychol Med, 49(4), 655-663. doi:10.1017/S0033291718001356
63. Passie, T., Halpern, J. H., Stichtenoth, D. O., Emrich, H. M., & Hintzen, A. (2008). The pharmacology of lysergic acid diethylamide: a review. CNS Neurosci Ther, 14(4), 295-314. doi:10.1111/j. 1755-5949.2008.00059.x
64. Perera, R. P., Wimalasena, D. S., & Wimalasena, K. (2003). Characterization of a series of 3-amino-2-phenylpropene derivatives as novel bovine chromaffin vesicular monoamine transporter inhibitors. J Med Chem, 46(13), 2599-2605. doi:10.1021/jm030004p
65. Rickli, A., Moning, O. D., Hoener, M. C., & Liechti, M. E. (2016). Receptor interaction profiles of novel psychoactive tryptamines compared with classic hallucinogens. Eur Neuropsychopharmacol, 26, 1327-1337.
66. Roseman, L., Nutt, D. J., & Carhart-Harris, R. L. (2017). Quality of acute psychedelic experience predicts therapeutic efficacy of psilocybin for treatment-resistant depression. Front Pharmacol, 8, 974. doi:10.3389/fphar.2017.00974
67. Ross, S., Bossis, A., Guss, J., Agin-Liebes, G., Malone, T., Cohen, B., . . . Schmidt, B. L. (2016). Rapid and sustained symptom reduction following psilocybin treatment for anxiety and depression in patients with life-threatening cancer: a randomized controlled trial. J Psychopharmacol, 30(12), 1165-1180. doi:10.1177/0269881116675512
68. Roth, G., Liepold, B., Mueller, S., & Bestmann, H. (2004). Further Improvements of the Synthesis of Alkynes from Aldehydes. Synthesis, 1, 59-62. doi: 10.1055/s-2003-44346
69. Sanches, R. F., de Lima Osorio, F., Dos Santos, R. G., Macedo, L. R., Maia-de-Oliveira, J. P., Wichert-Ana, L., . . . Hallak, J. E. (2016). Antidepressant Effects of a Single Dose of Ayahuasca in Patients With Recurrent Depression: A SPECT Study. J Clin Psychopharmacol, 36(1), 77-81. doi: 10.1097/jcp.0000000000000436
70. Schmid, Y., Enzler, F., Gasser, P., Grouzmann, E., Preller, K. H., Vollenweider, F. X., . . . Liechti, M. E. (2015). Acute effects of lysergic acid diethylamide in healthy subjects. Biol Psychiatry, 78(8), 544-553. doi:10.1016/j.biopsych.2014.11.015
71. Schmid, Y., Gasser, P., Oehen, P., & Liechti, M. E. (2021). Acute subjective effects in LSD- and MDMA-assisted psychotherapy. J Psychopharmacol, 35(4), 362-374. doi:10.1177/0269881120959604
72. Schmid, Y., Hysek, C. M., Simmler, L. D., Crockett, M. J., Quednow, B. B., & Liechti, M. E. (2014). Differential effects of MDMA and methylphenidate on social cognition. J Psychopharmacol, 28, 847-856.
73. Schmid, Y., & Liechti, M. E. (2018). Long-lasting subjective effects of LSD in normal subjects. Psychopharmacology (Berl), 235(2), 535-545. doi:10.1007/s00213-017-4733-3
74. Shulgin, A., & Shulgin, A. (1991). PiHKAL: a chemical love story (1 edn).
75. Shulgin, A. T., & Shulgin, A. (1997). TiHKAL the continuation: Berkeley: Transform Press.
76. Simmler, L., Buser, T., Donzelli, M., Schramm, Y., Dieu, L. H., Huwyler, J., . . . Liechti, M. E. (2013). Pharmacological characterization of designer cathinones in vitro. Br J Pharmacol, 168(2), 458-470. doi:10.1111/j.1476-5381.2012.02145.x
77. Snyder, S. H., Faillace, L., & Hollister, L. (1967). 2,5-dimethoxy-4-methyl-amphetamine (STP): a new hallucinogenic drug. Science, 158(3801), 669-670. doi:10.1126/science.158.3801.669
78. Trachsel, D., Lehmann, D., & Enzensperger, C. (2013). Phenethylemine: Von der Struktur zur Funktion (1 edn). Solothurn: Nachtschatten Verlag.
79. Vargas, M. V., Dunlap, L. E., Dong, C., Carter, S. J., Tombari, R. J., Jami, S. A., . . . Olson, D. E. (2023). Psychedelics promote neuroplasticity through the activation of intracellular 5-HT2A receptors. Science, 379(6633), 700-706. doi:10.1126/science.adf0435
80. Velmourougane, G., Harbut, M. B., Dalal, S., McGowan, S., Oellig, C. A., Meinhardt, N., . . . Greenbaum, D. C. (2011). Synthesis of new (-)-bestatin-based inhibitor libraries reveals a novel binding mode in the S1 pocket of the essential malaria M1 metalloaminopeptidase. J Med Chem, 54 (6), 1655-1666. doi:10.1021/jm101227t
81. Verrico, C. D., Miller, G. M., & Madras, B. K. (2007). MDMA (ecstasy) and human dopamine, norepinephrine, and serotonin transporters: implications for MDMA-induced neurotoxicity and treatment. Psychopharmacology, 189(4), 489-503. Retrieved from http://www.ncbi.nlm.nih.gov/entrez/query.fcgi?cmd=Retrieve&db=PubMed&dopt=Citatio n&list_uids=16220332
82. Vollenweider, F. X., Vontobel, P., Hell, D., & Leenders, K. L. (1999). 5-HT modulation of dopamine release in basal ganglia in psilocybin-induced psychosis in man—a PET study with [11C]raclopride. Neuropsychopharmacology, 20(5), 424-433. doi:10.1016/S0893-133X(98)00108-0

What is claimed is:

1. A composition comprising a compound represented by FIGS. 1A-1E, characterized in that:
R_{α1} and R_{α2} are, independently and in any combination, hydrogen, deuteron, C₁-C₆ saturated and unsaturated alkyl optionally deuterated or fluorinated, C₃-C₆ saturated and unsaturated cycloalkyl-(C₁-C₆)alkyl optionally deuterated or fluorinated, and R_{α1} and R_{α2} can be combined to form a cyclic moiety such as cycloalkyl, oxacycloalkyl, thiacycloalkyl or azacycloalkyl, which can be further substituted in any combination with deuteron, fluorine, alkyl, alkenyl or alkynyl substituents, and
R1, R2, R5 and R6 are, independently and in any combination hydrogen, deuteron, or fluorine; or
C₁-C₆ branched or unbranched alkyl with the alkyl optionally and independently substituted with F₁-F₁₃ fluorine and/or D₁-D₁₃ deuteron substituents; or
C₃-C₆ cycloalkyl optionally and independently substituted with one or more substituents such as F₁-F₁₃ fluorine and/or D₁-D₁₁ deuteron and/or C₁-C₂ alkyl; or
(C₃-C₆ cycloalkyl)-C₁-C₂ branched or unbranched alkyl optionally and independently substituted with one or more substituents such as F₁-F₁₇ fluorine and/or D₁-D₁₇ deuteron and/or C₁-C₂ alkyl; or
C₂-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₆ branched or unbranched alkynyl where any of the carbons of the branched or unbranched alkynyl substituent is optionally substituted independently and in any combination with one or more C₁-C₂ alkyl, with F₁-F₉ fluorine, with D₁-D₉ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₃-C₆ branched or unbranched alkoxyalkyl, alkoxyalkenyl or alkoxyalkynyl optionally and independently substituted with F₁-F₁₃ fluorine and/or D₁-D₁₃ deuteron substituents; or
any halogen; or
a nitrogen-containing substituent such as CN or NO₂; and furthermore
R3 and R4 are, independently and in any combination, hydrogen; or C₁-C₃ branched or unbranched alkyl with the alkyl optionally and independently substituted with F₁-F₇ fluorine and/or D₁-D₇ deuteron substituents or with aryl or heteroaryl bearing no up to any number of ether, thioether, halogen, alkyl, fluorinated alkyl, alkenyl, alkynyl or nitrogen-containing substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₆ branched or unbranched alkyl optionally and independently substituted with one or more substituents such as F₁-F₁₅ fluorine and/or D₁-D₁₅ deuteron and/or C₁-C₂ alkyl; or
C₃-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₅ fluorine, with D₁-D₁₅ deuteron, with C₂ alkenyl; or
C₃-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl; or are combined to form a cyclic moiety such as C₃-C₆ cycloalkyl, oxacycloalkyl, thiacycloalkyl, or azacycloalkyl, which can be further substituted in any combination with deuteron, fluorine, alkyl, alkenyl or alkynyl substituents; and furthermore
A represents aryl, wherein the aryl is independently di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro, iodo; or
A represents an indole-, benzo [1,3]dioxolyl-, 1,3-benzoxathiolyl-, a 1,3-benzodithiolyl-, a 2,3-dihydrobenzofuranyl, a 2,3-dihydrobenzo [b]thienyl- or a benzothioenyl group wherein said groups are independently attached and are independently unsubstituted, mono-, di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro or iodo;
and further characterized in that any non-protic hydrogen in said composition can be replaced by a deuteron or a fluorine in any combination.

2. The composition according to claim 1, wherein said compound is chosen from the group consisting of 2-(1,3-benzodioxol-5-yl)-3-(methylamino)butene (BME-MDMA, compound 8, FIG. 2A), 1-(1,3-benzodioxol-5-yl)but-3-en-2-amine (DH-BDB, compound 24, FIG. 2B), N-methyl-1-(1,3-benzodioxol-5-yl)but-3-en-2-amine (DH-MBDB, compound 25, FIG. 2C), 1-(1,3-benzodioxol-5-yl)but-3-yn-2-amine (DDH-BDB, compound 27, FIG. 2D), N-methyl-1-(1,3-benzodioxol-5-yl)but-3-yn-2-amine (DDH-MBDB, compound 28, FIG. 2E), 1-(4-bromo-2,5-dimethoxyphenyl)but-3-en-2-amine (DH-4C-B, compound 35, FIG. 2F), 1-(4-bromo-2,5-dimethoxyphenyl)but-3-yn-2-amine (DDH-4C-B, compound 37, FIG. 2G) and 1-(indol-3-yl)but-3-en-2-amine (DH-α-ET, compound 43, FIG. 2H).

3. The composition of claim 1, wherein said compound is a free base.

4. The composition of claim 1, wherein said compound is a salt thereof.

5. The composition of claim 4, wherein said compound is a hydrochloride salt or a fumarate salt thereof.

6. The composition of claim 5, wherein said compound is a pharmacologically acceptable acid addition salt thereof.

7. The composition of claim 1, wherein said compound is chosen from the group consisting of a racemate, a single enantiomer, a diastereomer, and a mixture of enantiomers or diastereomers in any ratio, a single and a mixture of E or Z configurational isomer in any ratio, a single and a mixture of cis or trans configurational isomer in any ratio, or combinations thereof.

8. The composition of claim 1, wherein said compound includes a prodrug.

9. A method of changing neurotransmission, including the steps of:
administering a pharmaceutically effective amount of composition to a mammal of a compound represented by FIGS. 1A-1E, characterized in that:
R_{α1} and R_{α2} are, independently and in any combination, hydrogen, deuteron, C₁-C₆ saturated and unsaturated alkyl optionally deuterated or fluorinated, C₃-C₆ saturated and unsaturated cycloalkyl-(C₁-C₆)alkyl optionally deuterated or fluorinated, and R_{α1} and R_{α2} can be combined to form a cyclic moiety such as cycloalkyl, oxacycloalkyl, thiacycloalkyl or azacycloalkyl, which can be further substituted in any combination with deuteron, fluorine, alkyl, alkenyl or alkynyl substituents, and
R1, R2, R5 and R6 are, independently and in any combination hydrogen, deuteron, or fluorine; or
C₁-C₆ branched or unbranched alkyl with the alkyl optionally and independently substituted with F₁-F₁₃ fluorine and/or D₁-D₁₃ deuteron substituents; or
C₃-C₆ cycloalkyl optionally and independently substituted with one or more substituents such as F₁-F₁₁ fluorine and/or D₁-D₁₁ deuteron and/or C₁-C₂ alkyl; or
(C₃-C₆ cycloalkyl)-C₁-C₂ branched or unbranched alkyl optionally and independently substituted with one or more substituents such as F₁-F₁₇ fluorine and/or D₁-D₁₇ deuteron and/or C₁-C₂ alkyl; or
C₂-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₆ branched or unbranched alkynyl where any of the carbons of the branched or unbranched alkynyl substituent is optionally substituted independently and in any combination with one or more C₁-C₂ alkyl, with F₁-F₉ fluorine, with D₁-D₉ deuteron, with C₂ alkenyl or with aryl or heteroaryl bearing no up to any number of ether, thioether, halogen, alkyl, fluorinated alkyl, alkenyl, alkynyl or nitrogen-containing substituents; or C₃-C₆ branched or unbranched alkoxyalkyl, alkoxyalkenyl or alkoxyalkynyl optionally and independently substituted with F₁-F₁₃ fluorine and/or D₁-D₁₃ deuteron substituents; or
any halogen; or
a nitrogen-containing substituent such as CN or NO₂; and furthermore
R3 and R4 are, independently and in any combination, hydrogen; or C₁-C₃ branched or unbranched alkyl with the alkyl optionally and independently substituted with F₁-F₇ fluorine and/or D₁-D₇ deuteron substituents or with aryl or heteroaryl bearing no up to any number of ether, thioether, halogen, alkyl, fluorinated alkyl, alkenyl, alkynyl or nitrogen-containing substituents; or
C₃-C₆ cycloalkyl optionally and independently substituted with one or more substituents such as F₁-F₁₅ fluorine and/or D₁-D₁₅ deuteron and/or C₁-C₂ alkyl; or
(C₃-C₆ cycloalkyl)-C₁-C₆ branched or unbranched alkyl optionally and independently substituted with one or more substituents such as F₁-F₁₅ fluorine and/or D₁-D₁₅ deuteron and/or C₁-C₂ alkyl; or
C₃-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₅ fluorine, with D₁-D₁₅ deuteron, with C₂ alkenyl; or
C₃-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl; or
are combined to form a cyclic moiety such as C₃-C₆ cycloalkyl, oxacycloalkyl, thiacycloalkyl or azacycloalkyl, which can be further substituted in any combination with deuteron, fluorine, alkyl, alkenyl or alkynyl substituents; and furthermore
A represents aryl, wherein the aryl is independently di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro, iodo;
A represents an indole-, benzo [1,3]dioxolyl-, 1,3-benzoxathiolyl-, a 1,3-benzodithiolyl-, a 2,3-dihydrobenzofuranyl, a 2,3-dihydrobenzo [b]thienyl- or a benzothioenyl group wherein said groups are independently attached and are independently unsubstituted, mono-, di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro or iodo;
and further characterized in that any non-protic hydrogen in the composition can be replaced by a deuteron or a fluorine in any combination, increasing serotonin 5-HT2A receptor or monoamine transporter interaction in the mammal; and
inducing psychoactive effects in the mammal.

10. The method of claim 9, wherein the compound is chosen from the group consisting of 2-(1,3-benzodioxol-5-yl)-3-(methylamino)butene (BME-MDMA, compound 8, FIG. 2A), 1-(1,3-benzodioxol-5-yl)but-3-en-2-amine (DH-BDB, compound 24, FIG. 2B), N-methyl-1-(1,3-benzodioxol-5-yl)but-3-en-2-amine (DH-MBDB, compound 25, FIG. 2C), 1-(1,3-benzodioxol-5-yl)but-3-yn-2-amine (DDH-BDB, compound 27, FIG. 2D), N-methyl-1-(1,3-benzodioxol-5-yl)but-3-yn-2-amine (DDH-MBDB, compound 28, FIG. 2E), 1-(4-bromo-2,5-dimethoxyphenyl)but-3-en-2-amine (DH-4C-B, compound 35, FIG. 2F), 1-(4-bromo-2,5-dimethoxyphenyl)but-3-yn-2-amine (DDH-4C-B, compound 37, FIG. 2G) and 1-(indol-3-yl)but-3-en-2-amine (DH- α-ET, compound 43, FIG. 2H).

11. The method of claim 9, wherein said compound is a free base.

12. The method of claim 9, wherein said compound is a salt thereof.

13. The method of claim 12, wherein said compound is a hydrochloride salt or a fumarate salt thereof.

14. The method of claim 13, wherein said compound is a pharmacologically acceptable acid addition salt thereof.

15. The method of claim 9, wherein said compound includes a prodrug.

16. The method of claim 9, wherein the compound is chosen from the group consisting of a racemate, a single enantiomer, a diastereomer, and a mixture of enantiomers or diastereomers in any ratio, a single and a mixture of E or Z configurational isomer in any ratio, a single and a mixture of cis or trans configurational isomer in any ratio, or combinations thereof.

17. The method of claim 9, wherein the pharmacological and psychoactive effects include enhancing cognition and/or mood, psychedelic or entactogenic effects having intensity, effect quality, or duration of effect in a mammal in comparison to that of DOM, mescaline, LSD, psilocybin, or MDMA.

18. The method of claim 9, wherein the compound is administered to mammals for substance-assisted psychotherapy.

19. The method of claim 9, wherein the compound is administered to allow for changing dose potency in comparison to DOM or MDMA.

20. The method of claim 9, wherein the compound is administered to allow for tailoring and treatment individualization to the mammal's therapeutic need.

21. The method of claim 9, wherein the mammal is a human.

22. The method of claim 9, further including the step of treating a medical disorder chosen from the group consisting of post-traumatic stress disorder, social anxiety, autism spectrum disorder, substance use disorder, depression, psychotic symptoms, Parkinson's disease, cognition disorders, anxiety disorder, anxiety with life-threatening disease, personality disorder including narcistic or antisocial personality disorder, obsessive compulsive disorder, attention-deficit/hyperactive disorder, eating disorder, and pain.

23. The method of claim 9, further including the step of using the composition for a therapy chosen from the group consisting of couple therapy, enhancement of psychotherapy, and enhancing therapeutic alliance in psychotherapy of patients or neurotic/healthy subjects.

24. A method of treating a patient having adverse reactions to psychedelics or entactogens, including the steps of:
administering a pharmaceutically effective amount of composition to the patient of a compound represented by FIGS. 1A-1E, characterized in that
R_{α1} and R_{α2} are, independently and in any combination, hydrogen, deuteron, C₁-C₆ saturated and unsaturated alkyl optionally deuterated or fluorinated, C₃-C₆ saturated and unsaturated cycloalkyl-(C₁-C₆)alkyl optionally deuterated or fluorinated, and R_{α1} and R_{α2} can be combined to form a cyclic moiety such as cycloalkyl, oxacycloalkyl, thiacycloalkyl or azacycloalkyl, which can be further substituted in any combination with deuteron, fluorine, alkyl, alkenyl or alkynyl substituents, and
R1, R2, R5 and R6 are, independently and in any combination hydrogen, deuteron, or fluorine; or
C₁-C₆ branched or unbranched alkyl with the alkyl optionally and independently substituted with F₁-F₁₃ fluorine and/or D₁-D₁₃ deuteron substituents; or
C₃-C₆ cycloalkyl optionally and independently substituted with one or more substituents such as F₁-F₁₁ fluorine and/or D₁-D₁₁ deuteron and/or C₁-C₂ alkyl; or (C₃-C₆ cycloalkyl)-C₁-C₂ branched or unbranched alkyl optionally and independently substituted with one or more substituents such as F₁-F₁₇ fluorine and/or D₁-D₁₇ deuteron and/or C₁-C₂ alkyl; or
C₂-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₂-C₆ branched or unbranched alkynyl where any of the carbons of the branched or unbranched alkynyl substituent is optionally substituted independently and in any combination with one or more C₁-C₂ alkyl, with F₁-F₉ fluorine, with D₁-D₉ deuteron, with C₂ alkenyl or with aryl or heteroaryl bearing no up to any number of ether, thioether, halogen, alkyl, fluorinated alkyl, alkenyl, alkynyl or nitrogen-containing substituents; or
C₃-C₆ branched or unbranched alkoxyalkyl, alkoxyalkenyl or alkoxyalkynyl optionally and independently substituted with F₁-F₁₃ fluorine and/or D₁-D₁₃ deuteron substituents; or
any halogen; or
a nitrogen-containing substituent such as CN or NO₂; and furthermore
R3 and R4 are, independently and in any combination, hydrogen; or C₁-C₃ branched or unbranched alkyl with the alkyl optionally and independently substituted with F₁-F₇ fluorine and/or D₁-D₇ deuteron substituents or with aryl or heteroaryl bearing no up to any number of ether, thioether, halogen, alkyl, fluorinated alkyl, alkenyl, alkynyl or nitrogen-containing substituents; or
C₃-C₆ cycloalkyl optionally and independently substituted with one or more substituents such as F₁-F₁₅ fluorine and/or D₁-D₁₅ deuteron and/or C₁-C₂ alkyl; or (C₃-C₆ cycloalkyl)-C₁-C₆ branched or unbranched alkyl optionally and independently substituted with one or more substituents such as F₁-F₁₅ fluorine and/or D₁-D₁₅ deuteron and/or C₁-C₂ alkyl; or
C₃-C₆ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₅ fluorine, with D₁-D₁₅ deuteron, with C₂ alkenyl; or
C₃-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl; or
are combined to form a cyclic moiety such as C₃-C₆ cycloalkyl, oxacycloalkyl, thiacycloalkyl or azacycloalkyl, which can be further substituted in any combination with deuteron, fluorine, alkyl, alkenyl or alkynyl substituents; and furthermore
A represents aryl, wherein the aryl is independently di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro, iodo; or
A represents an indole-, benzo [1,3] dioxolyl-, 1,3-benzoxathiolyl-, a 1,3- benzodithiolyl-, a 2,3-dihydrobenzofuranyl, a 2,3-dihydrobenzo [b] thienyl- or a benzothioenyl group wherein said groups are independently attached and are independently unsubstituted, mono-, di-, tri-, tetra- or penta-substituted, wherein the substituents are independently and in any combination D₀-D₁₃ deuterated or F₀-F₁₃ fluorinated and selected from the group consisting of C₁-C₅ alkyl, C₂-C₅ alkenyl, C₂-C₅ alkynyl, C₁-C₅ alkoxy, C₂-C₅ alkenyloxy, C₃-C₅ alkynyloxy, C₁-C₅ alkylthio, C₂-C₅ alkenylthio, C₃-C₅ alkynylthio, C₃-C₅ cycloalkyl, C₃-C₅ cycloalkoxy, C₃-C₅ cycloalkylthio, C₄-C₅ cycloalkenyl, C₄-C₅ cycloalkenyloxy, C₄-C₅ cycloalkenylthio nitrile, nitro, fluoro, bromo, chloro or iodo;
and further characterized in that any non-protic hydrogen in the composition can be replaced by a deuteron or a fluorine in any combination; and
avoiding adverse effects present with psychedelics or entactogens.

25. The method of claim 24, wherein the compound is chosen from the group consisting of 2-(1,3-benzodioxol-5-yl)-3-(methylamino)butene (BME-MDMA, compound 8, FIG. 2A), 1-(1,3-benzodioxol-5-yl)but-3-en-2-amine (DH-BDB, compound 24, FIG. 2B), N-methyl-1-(1,3-benzodioxol-5-yl)but-3-en-2-amine (DH-MBDB, compound 25, FIG. 2C), 1-(1,3-benzodioxol-5-yl)but-3-yn-2-amine (DDH-BDB, compound 27, FIG. 2D), N-methyl-1-(1,3-benzodioxol-5-yl)but-3-yn-2-amine (DDH-MBDB, compound 28, FIG. 2E), 1-(4-bromo-2,5-dimethoxyphenyl)but-3-en-2-amine (DH-4C-B, compound 35, FIG. 2F), 1-(4-bromo-2,5-dimethoxyphenyl)but-3-yn-2-amine (DDH-4C-B, compound 37, FIG. 2G) and 1-(indol-3-yl)but-3-en-2-amine (DH-α-ET, compound 43, FIG. 2H).

26. The method of claim 24, wherein the adverse effects are chosen from the group consisting of less anxiety, less cardio-stimulant effects, less thermogenesis, less adverse effects, less nausea, and combinations thereof.

27. The method of claim 26, further including the step of providing more positive effects than other psychedelics or entactogens.

28. The method of claim 27, wherein the positive effects are chosen from the group consisting of more overall positive effects, more or less perceptual effects, more emotional effects, and combinations thereof.

29. The method of claim 24, further including the step of providing a shorter duration of action than with other psychedelics or entactogens such as LSD, DOM, or MDMA.
